SEQUENCE LISTING (8 sequences)

SEQ ID NO: 1 - DNA, 5454 bp, Escherichia coli
  Expression plasmid pPA-GBP, for His6-protein A-GBP. Vector is pQE-80L (Qiagen). CDS for Protein A (nucleotides 160-528) is from pEZZ18 (Amersham). CDS for GBP (nucleotides 565 -858) is from pSB3053 from (Brown, Nat. Biotechnol. 15269-272, 1997).
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccggttctggtgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaataagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 2 - DNA, 5448 bp, Escherichia coli
  Expression plasmid pStreptavidin-GBP for His6- streptavidin-GBP. pQE-80L vector(Qiagen). CDS core streptavidin (169-531), (Chilkoti et al., Proc Natl Acad Sci USA 921754-1758, 1995). CDS GBP (559-852), pSB3053, (Brown, Nat. Biotechnol. 15269 -272, 1997).
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccggttctggtggccatatggctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagctaacgcgtggaaatccaccctggttggtcacgacaccttcaccaaagtttcgagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaataagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 3 - DNA, 1182 bp, Escherichia coli
  Sequence shows CDS portion (115-1293) of the expression plasmid, pPA-GBP-PA for fusion protein His6-protein A -GBP-protein A. The total length of the plasmid is 5859 base pairs. The origin of the basic vector and other CDSs is the same as pPA-GBP.
atgagaggatcgcatcaccatcaccatcacggatccggttctggtgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtggcggatccggttctggtgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctctggtggctaa

SEQ ID NO: 4 - DNA, 1170 bp, Escherichia coli
  CDS portion (115-1281) of the expression plasmid, pStrept-GBP-Strept (total length 5833 base pairs) for fusion protein His6-streptavidin-GBP-streptavidin. Vector and other CDSs are the same as pPA-GBP and pStreptavidin-GBP.
atgagaggatcgcatcaccatcaccatcacggatccggttctggtggccatatggctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagctaacgcgtggaaatccaccctggttggtcacgacaccttcaccaaagtttcgagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggatctggttctggtggccatatggctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagctaacgcgtggaaatccaccctggttggtcacgacaccttcaccaaagtttcgagctcaagcttaattagctga

SEQ ID NO: 5 - DNA, 1176 bp, Escherichia coli
  CDS portion (115-1287) of the expression plasmid, pPA-GBP-Streptavidin (total length 5839 base pairs) for fusion protein His6-PA-GBP-streptavidin. Vector and other CDSs are the same as pPA-GBP and pStreptavidin-GBP.
atgagaggatcgcatcaccatcaccatcacggatccggttctggtgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggatctggttctggtggccatatggctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagctaacgcgtggaaatccaccctggttggtcacgacaccttcaccaaagtttcgagctcaagcttaattagctga

SEQ ID NO: 6 - DNA, 1176 bp, Escherichia coli
  CDS portion (115-1287) of the expression plasmid, pStreptavidin-GBP-PA (total length 5853 base pairs) for fusion protein His6-streptavidin-GBP-PA. Vector and other CDSs are the same as pPA-GBP and pStreptavidin-GBP.
atgagaggatcgcatcaccatcaccatcacggatccggttctggtggccatatggctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagctaacgcgtggaaatccaccctggttggtcacgacaccttcaccaaagtttcgagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtggcggatccggttctggtgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctctggtggctaa

SEQ ID NO: 7 - DNA, 393 bp, Escherichia coli
  CDS portion (115-504) of the expression plasmid, pGBP (total length 5073 base pairs) for fusion protein His6-GBP. Vector and other CDSs are the same as pPA-GBP.
atgagaggatcgcatcaccatcaccatcacggatccggaggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

SEQ ID NO: 8 - DNA, 741 bp, Escherichia coli
  CDS portion (115-852) of the expression plasmid, pGBP-GBP (total length 5421 base pairs) for fusion protein His6-GBP-GBP. Vector and other CDSs are the same as pPA-GBP.
atgagaggatcgcatcaccatcaccatcacggatccggaggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtggtggaagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

(19) United States
(12) Patent Application Publication
(10) Pub. No.: US 2005/0106625 A1
(43) Pub. Date: May 19, 2005

(54) Recombinant fusion proteins with high affinity binding to gold and applications thereof

(75) Inventors: Richard G. Woodbury, Seattle, WA (US); Theo deVos, Seattle, WA (US); Meher Irani, Seattle, WA (US)

Correspondence Address: BioHesion Incorporated, 1208 NE 100th Street, Seattle, WA, 98125, US

(73) Assignee: BioHesion Incorporated, Seattle, WA (US)

(21) Appl. No.: 10/671,995

(22) Filed: Sep. 26, 2003

(51) Int. Cl. G01N 33/53; C07H 21/04; C07K 7/08

(52) U.S. Cl. 435/7.1; 435/69.1; 435/320.1; 435/325; 530/327; 536/23.5

(57) ABSTRACT

The present invention provides a method to firmly attach any polypeptide to a gold surface regardless of its intrinsic gold-binding properties. The method describes the production of recombinant fusion proteins consisting of polypeptides of interest and a high affinity gold binding peptide consisting of 1 to 7 repeats of a unique amino acid sequence. By this method, many biologically active polypeptides lacking intrinsic gold-binding properties can be firmly attached to gold surfaces. The disclosure includes evidence that fusion proteins containing the gold-binding sequences provide superior stability and activity compared to similar molecules lacking the tag when used to construct biosensors. The invention provides a method that is a significant improvement over existing chemical and physical adsorption protocols to attach polypeptides to gold and, therefore, can provide benefits to many applications utilizing gold.

The research described in the Examples appearing in this nonprovisional patent application was supported by a Phase I Small Business Innovative Research grant #1 R43 CA101579-01, entitled “Development of Novel Gold Binding Fusion Proteins”. All rights to any inventions are retained by BioHesion, Incorporated, Seattle, Wash.

A sequence listing is attached as an appendix to this application.

### FIELD OF THE INVENTION

The present invention relates to the production of fusion proteins containing a unique polypeptide sequence with the capacity to bind gold surfaces with high affinity. Fusion partners can be encoded in recombinant molecules to introduce specific binding or enzymatic activity to surfaces. The invention relates specifically to production of unique recombinant fusion proteins to support applications in all fields utilizing gold including, but not limited to clinical diagnostic testing, laboratory research, biosensor develpment, proteomics, drug testing, and biomaterial.

### BACKGROUND OF THE INVENTION

Robust attachment of proteins and other macromolecules, e.g., recognition or affinity-binding molecules or enzymes, to a surface such as gold is an essential step in implementing a variety of technologies targeting numerous applications in clinical diagnostics, laboratory research, biosensors, biomaterials, proteomics, and drug discovery/evaluation fields. Gold is an excellent material for introducing surface functionality via the attachment of proteins or other macromolecules because of the metal's chemical inertness, electrical conductivity, surface uniformity and stability, biologic compatibility/low toxicity and other properties. Gold's chemical inertness, however, limits the ability to prepare functional surfaces to just a few proteins or other macromolecules that produce stable biofilms when adsorbed directly onto a clean gold surface. For example, certain classes of immunoglobulin, streptavidin, protein A and certain proteins or peptides with basic charges passively adsorb to gold at pH 6 to 8 in appropriate buffers containing relatively low concentrations of salts (Scopsi, et al., J. Histochem Cytochem 34:1469-1475, 1986).

Many proteins and macromolecules of interest, however, do not adsorb readily to gold with subsequent retention of biological activity. Whether or not a particular molecule binds to gold depends on certain molecular properties and solvent conditions. Most important, the surface charge of proteins and other molecules appears to affect the interaction with gold, favoring those molecules with basic charges (Scopsi, et al., J. Histochem Cytochem 34:1469-1475, 1986). Therefore, the current methods of direct adsorption of proteins and other macromolecules to gold are successful only for a relatively few examples of the large number of molecules of interest with commercial potential. The method of direct adsorption of molecules to gold, therefore, severely impedes the development of novel applications in all fields utilizing gold. Improved methods are needed to attach many different classes of proteins, other macromolecules and small molecules to gold.

Another disadvantage of current methods for direct physical adsorption of proteins, other macromolecules and small molecules to gold is that most of the resulting complexes can be unstable. For example, complexes of immunoglobulin or protein A and gold can dissociate in aqueous solution prior to, during, and following intended applications or can be displaced from gold in the presence of other proteins and macromolecules during applications (Horisberger and Clerc, Histochemistry 82:219-223, 1985; Geoghegan, J Histochem Cytochem 36:401-407, 1988). Such instability can lead to inconsistent results for test samples, limit the number of potential applications, and result in gold-protein complexes that have short storage lives. Improved methods are needed to increase the stability of gold complexes with proteins and other molecules of interest.

Another disadvantage of direct adsorption of proteins, other macromolecules, and small molecules to gold is that the attachment can be a random process in regard to which surface of the molecule binds gold. Random attachment can result in inefficient orientation or presentation of active sites of molecules that interact with target molecules or substrates in solution. Improper orientation of active sites on a significant proportion of molecules on gold can reduce the sensitivity and utility of molecule-gold complexes in applications. Improved methods are needed to control the orientation of molecules attached to gold to increase access of target molecules or substrates to the active sites of the attached molecule.

Another disadvantage of direct adsorption of molecules, especially proteins, to gold is the frequent occurrence of molecular denaturation or inactivation when molecules in solution bind directly to surfaces (Engel, et al., J Biol Chem 277:10922-10930, 2002; Postel, et al., J. Colloid Interfaces Sci 266:74-81, 2003). Denaturation of proteins, in particular, can lead to waste of valuable proteins and can increase non-specific binding of materials to the surface causing fouling. Improved methods are needed to reduce the extent that proteins and other macromolecules denature on gold surfaces.

Another disadvantage of direct adsorption of molecules to gold that limits development of commercial applications in all fields utilizing gold is that only large molecules such as proteins, proteoglycans, or structures such as membrane-bound lipids typically bind well to gold. With the exception of sulfur-containing compounds and certain salts and other ions, most small molecules have weak affinity to gold. Consequently, many small polypeptides incuding hormones, antigens, steroid-based hormones, other receptor ligands, pesticides, other environmental toxins, or the like cannot be attached directly to gold. Methods exist for the covalent attachment of desired small molecules linked via reactive groups in foundation layers of bovine serum albumin or thiol compounds that can bind gold (Elkind, et al., Sensors & Actuators B, 54:182-190, 1999; Bain, et al., J. Am Chem. Soc. 111:321-335, 1989). Such approaches, however, are inefficient for the general reasons discussed above for proteins.

Additional disadvantages are that small molecules of interest typically contain few or no suitable reactive groups for attachment to foundation layers and many small molecules are inactive following covalent attachment to a foundation layer. Improved methods are needed to attach small polypeptides and other molecules to gold with the retention of activity.

The general ineffectiveness of current methods for direct adsorption of proteins and other macromolecules to gold as described above has stimulated effort to develop improved methods for introducing active molecules to gold surfaces. In one process, alkanethiol monolayers with reactive groups at the distil end of the molecules can be introduced on gold to allow attachment of molecules of interest at the surface (Bain, et al., J. Am. Chem. Soc. 111:321-335, 1989; Lofas and Johnsson, J. Am. Chem. Soc. Commun. 1526-1528, 1990). In this manner, the desired molecules typically do not interact directly with the gold surface. However, such biofilms can be unstable in complex solutions or whenever sulfur-containing compounds are present and these biofilms have limited utility in applications outside of the laboratory (Schlenoff, et al., J. Am. Chem. Soc. 117:12528-12536, 1995; Melendez, et al., Sensors & Actuators B, 35, 36:212-216, 1996).

In the field of surface plasmon resonance (SPR) biosensors, in particular, BIAcore (Sweden) achieved improvements in the stability and utility of alkanethiol monolayers on gold through the covalent attachment of a layer of high molecular weight dextran to the monolayer (Jonsson, et al., BioTechniques 11:620-627, 1991). The dextran hydrogel contains reactive groups for attaching proteins and other macromolecules in a favorable hydrophilic environment. The introduction of the dextran layer also stabilizes the alkanethiol monolayer on gold and helps reduce non-specific binding to gold. The BIAcore technology supports commercial instruments used entirely for research purposes where test conditions can be strictly controlled. However, analysis of complex clinical and environmental samples remains problematic for BIAcore's instruments because the sulfur-gold linkage is labile when samples contain sulfur-based compounds, including proteins with surface cysteines. Additionally, while BIAcore's technology reduces non-specific binding during testing of simple, well-defined laboratory solutions, non-specific binding precludes testing of many environmental, clinical, industrial and other complex samples with BIAcore instruments.

The discovery of a gold-binding peptide, GBP, (Brown, Nat. Biotechnol. 15:269-272, 1997) and studies by Woodbury and coworkers (Woodbury, et al., Sensors & Bioelectronics, 13:1117-1126, 1998) led to an invention disclosing a chemical method to link recognition proteins to gold via GBP to construct SPR biosensors (U.S. Pat. No. 6,239,255). The process requires binding a recombinant GBP-alkaline phosphatase chimera to the gold surface, removing the alkaline phosphatase domain with proteases, activating chemical groups on the GBP domain that remains attached to gold, and introducing the desired recognition protein for covalent attachment to the GBP foundation. However, the process is tedious, inefficient and not readily applicable to constructing arrays consisting of many different proteins or other macromolecules that can require numerous, different chemical procedures to achieve attachment of all molecules of interest. In addition, the chemical method disclosed in U.S. Pat. No. 6,239,255 does not teach how the orientation of molecules, with the exception of certain classes of immunoglobulins, can be controlled to retain high specific activity when they are attached to gold. Further, the approach can have limited usefulness for applications utilizing colloidal gold that can be unstable under certain conditions required for the covalent attachment of molecules to reactive groups on GBP or other foundation layer.

There are additional disadvantages of conventional chemical approaches to achieve the covalent attachment of molecules to dextran, e.g., in BIAcore's approach (Jonsson, et al., BioTechniques 11:620-627, 1991), or to GBP (Woodbury, et al., Sensors & Bioelectronics, 13:1117-1126, 1998; U.S. Pat. No. 6,239,255), or to other reactive surfaces. As noted above, no single chemical approach can be used to attach all molecules of interest to surfaces. A determination of suitable attachment chemistry for each molecule is a haphazard and time-consuming process. In addition, attachment chemistries frequently render molecules inactive or otherwise adversely alter the properties of molecules. Certain macromolecules, due to chemical composition, inaccessibility of potential reactive groups, and/or tertiary structure are not amenable to modification by covalent linking chemistry.

In the case of valuable molecules available in minute quantities, conventional methods can fail to attach sufficient numbers of molecules to gold. Increasingly, advances in nanotechnology and array technology require greater control of molecular orientation of tiny amounts of material than is possible using current attachment chemistries. Novel applications utilizing colloidal gold can be developed, if the relatively few molecules that bind to this form of gold can be extended to any protein, other macromolecules, and small polypeptides and other molecules of interest. In all fields utilizing gold, application performance can be enhanced with increased sensitivity due to full accessibility of active sites of attached molecules to target and substrate molecules. Similarly, all fields utilizing gold will benefit from significant cost reduction by eliminating the inefficiencies, as described above, inherent in current methods to attach molecules to gold.

The invention described herein overcomes many disadvantages and inefficiencies associated with current methods to attach molecules of interest to gold surfaces. Implementation of the invention will significantly reduce the cost, effort and inefficiencies of existing applications in all fields utilizing gold. Additionally, the invention will facilitate the development of novel commercial applications not possible or anticipated using current methods.

### SUMMARY OF INVENTION

The invention described herein produces recombinant fusion proteins consisting of three components that in combination simplify the production, purification and attachment of desired polypeptides, other macromolecules and small molecules to any gold surface. When compared to the individual components alone, the combination of the three components comprising the invention act in synergy to improve the overall stable production, purification and applications of fusion proteins of the type disclosed, herein, in ways not obvious from the prior art.

Specifically, the invention encodes a gold-binding peptide (GBP) for the stable attachment of fusion proteins to any gold surface. A second component is a fusion partner consisting of any desired polypeptide with specific binding or enzyme activity. The inclusion of short, flexible amino acid sequences linking GBP and fusion partner domains facilitates optimum physical orientation of each domain to allow full expression of GBP and fusion partner activities. A third component consists of a specific polypeptide affinity tag, e.g., polyhistidine (His₆-tag), that permits rapid purification of the fusion protein in essentially one step. Rapid purification from cellular extracts or secretions can minimize proteolytic degradation typically associated with the expression of fusion proteins. Without the presence of the affinity tag in fusion proteins, each fusion protein would require its own purification scheme that can be a costly, time-consuming endeavor accompanied by a multitude of difficulties.

The invention, therefore, eliminates all of the disadvantages of current methods for the attachment of proteins and small polypeptides to gold by transferring the gold-binding process to a polypeptide domain designed for this purpose. Further, the invention provides a rapid, one-step purification procedure that can be used for all fusion proteins of the type disclosed, herein.

The invention also provides, when desired, specific chemical or enzyme cleavage sites in the linking amino acid sequences between domains to allow the physical separation of fusion partner domains.

The invention provides for plasmid expression systems in bacterial, yeast, insect, and mammalian cell lines for the production of fusion proteins whereby GBP is placed at the amino terminus, internally, or at the carboxyl terminus of any other polypeptide. In certain embodiments, fusion partners of GBP can be, but are not limited to, Protein A or Protein G; or streptavidin; or single-chain antibodies; or enzymes such as glucose oxidase or horseradish peroxidase; or metallothionein; or receptors; or peptides suitable for the introduction of biotin; or any other affinity binding polypeptide. Fusion partners can be polypeptides that possess high affinity to bacteria or secreted products of bacteria. Fusion partners can be polypeptides that have high affinity to viruses or parasites. Fusion partners can be small polypeptide hormones such as insulin or angiotensin, or vasoactive or neuroactive molecules that interact with receptors. Other examples of small polypeptides that can be fusion partners are polypeptide epitopes recognized by specific antibodies.

In certain embodiments, affinity binding molecules of interest that are not polypeptides, e.g., nucleic acids, carbohydrates, lipids, lectins, and small molecules can be attached to the fusion protein on gold via one or more fusion partners. Specific nucleic acids, for example, can be labeled with biotin and can subsequently bind with high affinity to fusion proteins containing streptavidin. Similarly, fusion partners can be polypeptides that bind other cofactors or small molecules, wherein said cofactors and small molecules are linked to non-polypeptide targets.

In certain embodiments, GBP-fusion partners can be polypeptides derived from screening, e.g., diverse phage libraries, for active molecules. Active polypeptides can include those with selective binding affinity to specific proteins, or other macromolecules, small organic or inorganic molecules, surfaces other than gold, cells, viruses, parasites, or any substance of interest.

In certain embodiments, fusion partners of GBP can be enzymes, for example, but not limited to, glucose oxidase or horseradish peroxidase that are used to construct monitoring devices to measure blood glucose levels in diabetics or other analytes.

In this disclosure, we establish that fusion partners can be attached at either end of the GBP domain, an observation not taught by the prior art. Thus, in other embodiments, the invention permits two or more copies of a desired fusion partner attached to a single GBP domain to increase the specific binding capacity or enzymatic activity of the fusion protein attached to gold. When permitted, multiple copies of fusion partners can be expressed in tandem. When tandem expression of fusion partners is not permitted, a minimum of two copies of fusion partner can be expressed by placing one at the amino-terminus and the other at the carboxy-terminus of a single GBP domain.

In certain embodiments, the invention permits the production of fusion proteins containing two or more distinct fusion partners with different activities. For example, a chimera can be produced containing streptavidin at one end of GBP and Protein A at the other end. In another example, a fusion protein with multiple function is one containing two distinct enzymes attached to GBP. In another example, a mixed-function fusion protein is one whereby one fusion partner, e.g., a single-chain antibody or receptor, can bind specific molecules present in low concentration. The increased concentration of specific molecules in the vicinity of the fusion protein can significantly improve the activity of a second fusion partner, e.g., an enzyme that utilizes the specific molecules as substrate when conditions are changed to release the specific molecules from the binding domain of the fusion protein.

These examples and others of multiple and mixed function fusion proteins containing GBP can be valuable commercial reagents to support existing and novel applications in all fields utilizing gold. In particular, multiple and mixed function fusion proteins can have utility when applied to clinical diagnostic testing, or “lab-on-a-chip” devices, or protein arrays, or nanotechnology-based devices, or other emerging fields utilizing gold.

The invention overcomes many of the disadvantages of current methods to produce, purify, and attach proteins to gold. In this disclosure, we establish several advantages of GBP-based chimeras, as provided by the invention, not anticipated by those skilled in the art. For example, the prior art teaches that immunoglobulins, streptavidin, protein A bind gold well and the direct adsorption of these proteins has been used as a general approach to introduce activity to gold surfaces (Scopsi, et al., J. Histochem Cytochem 34:1469-1475, 1986; Geoghegan, J Histochem Cytochem 36:401-407, 1988). The prior art does not teach, however, as disclosed, herein, that recombinant Streptavidin-GBP fusion is 5- to 10-fold more active in binding biotinylated molecules than is recombinant Streptavidin lacking the GBP domain when each are bound to gold.

The inventive conception has been reduced to practice by us with the plasmid expression and protein production/purification of His₆-protein A-GBP, His₆-streptavidin-GBP, His₆-protein A-GBP-protein A, His₆-streptavidin-GBP-streptavidin, His₆-protein A-GBP-streptavidin, His₆-streptavidin-GBP-protein A, His₆-GBP, and His₆-GBP-GBP. Two examples, His₆-protein A-GBP and His₆-streptavidin-GBP fusion proteins, have been characterized to establish full function and superior activity and the others currently are being characterized. A further understanding of the nature and advantages of the invention will become apparent from the detailed descriptions of these examples, other specific examples of the invention, and other information provided below.

### BRIEF DESCRIPTION OF THE DRAWINGS

FIG. 1 depicts a general scheme for constructing GBP fusion proteins with any polypeptide partner(s) whereby the GBP sequence is positioned at the amino terminus, internally, or at the carboxyl terminus of the recombinant molecule. The drawings represent the DNA sequence encoding the fusion protein portion of a plasmid vector that can be expressed in host cells.

FIG. 2 depicts the plasmid map of the expression vector, pPA-GBP, designed to produce His₆-protein A-GBP fusion protein in E. coli cells.

FIG. 3 depicts the plasmid map of the expression vector, pStreptavidin-GBP, designed to produce His₆-streptavidin-GBP fusion protein in E. coli cells.

FIG. 4 depicts the SDS-PAGE analyses of the production of recombinant proteins, His₆-protein A-GBP, His₆-streptavidin-GBP, and His₆-streptavidin in E. coli cells. See figure legend for details.

FIG. 5 depicts the SDS-PAGE analyses of the purification of recombinant proteins, His₆-protein A-GBP, His₆-streptavidin-GBP, and His₆-streptavidin from cell extracts facilitated via the His₆ tag binding to nickel resin columns. See figure legend for details.

FIG. 6 depicts the selective cleavage of protein A-GBP fusion protein at an inserted Asn-Gly bond. See figure legend for details.

FIG. 7 depicts the gold binding and antibody binding activities of His₆-protein A-GBP fusion protein on gold powder compared to these activities of native protein A on gold powder.

FIG. 8 depicts the gold binding and biotin-binding activities of His₆-streptavidin-GBP fusion protein and recombinant His₆-streptavidin (lacking the GBP domain) on gold powder.

FIG. 9 depicts how gold stabilizes the GBP domain of His₆-streptavidin-GBP in the presence of guanidine-HCl.

FIG. 10 depicts sensorgrams of analyses of SPR biosensors constructed with His₆-protein A-GBP fusion protein or native protein A. See figure legend for details.

FIG. 11 depicts sensorgrams of analyses of SPR biosensors constructed with recombinant His₆-streptavidin-GBP or His₆-streptavidin. See figure legend for details.

FIG. 12 depicts the plasmid map of the expression vector, pPA-GBP-PA, designed to produce His₆-protein A-GBP-protein A fusion protein in E. coli cells.

FIG. 13 depicts the plasmid map of the expression vector, pStrept-GBP-Strept, designed to produce His₆-streptavidin-GBP-streptavidin fusion protein in E. coli cells.

FIG. 14 depicts the plasmid map of the expression vector, pPA-GBP-Streptavidin, designed to produce His₆-protein A-GBP-streptavidin fusion protein in E. coli cells.

FIG. 15 depicts the plasmid map of the expression vector, pStreptavidin-GBP-PA, designed to produce His₆-streptavidin-GBP-PA fusion protein in E. coli cells.

FIG. 16 depicts the plasmid map of the expression vector, pGBP, designed to produce His₆-GBP (GBP monomer) fusion protein in E. coli cells.

FIG. 17 depicts the plasmid map of the expression vector, pGBP-GBP, designed to produce His₆-GBP-GBP (GBP dimer) fusion protein in E. coli cells.

FIG. 18 depicts a GBP-fusion protein bound to a gold surface. In this representation, the GBP sequence is fused to a single-chain antibody partner. The design of this system results in complete accessibility of analyte molecules, e.g., antigens, to the binding site of the GBP-fusion partner.

### DESCRIPTION OF THE SPECIFIC EMBODIMENTS

The invention described herein produces recombinant fusion proteins consisting of a unique GBP (Brown, Nat. Biotechnol. 15:269-272, 1997) consisting of 7 repeats of the 14 amino acid sequence, Met-His-Gly-Lys-Thr-Gln-Ala-Thr-Ser-Gly-Thr-Ile-Gln-Ser, and any desired polypeptide specifying activity, binding such fusion protein to a gold surface thereby introducing functionality to the surface. The invention provides the following improvements compared to existing methods:

No linking chemistry is required to attach desired polypeptides to GBP. This important benefit saves time, reagents and increases overall efficiency. With conventional methods different coupling chemistries can be required to attach distinct proteins to a GBP or other foundation layer. For example, when protein array chips are constructed with hundreds or thousands of unique proteins the complexity of many different linking chemistries, variable reaction rates and unequal protein coupling present formidable challenges to achieve functional uniformity on any single array and consistency among replicate arrays. The recombinant molecules provided by the present invention eliminate these technical difficulties and uncertainties by simplifying the entire surface derivitization process to a single, rapid step, i.e., the specific interaction of GBP and gold. Thus, the invention provides a method to achieve high uniformity and consistency in the manufacture of gold chips, colloidal gold, or any gold surface consisting of one or many distinct recognition or binding polypeptides or enzymes.

For certain fusion proteins, depending on application, there is no requirement to purify GBP or the desired protein prior to adsorbing them onto gold. The affinity and specificity of GBP to gold are sufficiently high, e.g., K_{D}=1.5×10⁻¹⁰M (Brown, Nat. Biotechnol. 15:269-272, 1997) to allow specific interaction in crude preparations containing many irrelevant proteins and other macromolecules.

The one to one relationship of GBP to fusion partner in the proposed recombinant molecules enables one to construct uniform foundation layers containing high densities of functional protein. This can increase the sensitivity of detection in applications compared to that provided by conventional chemical attachment methods.

The recombinant molecules can be constructed to orient recognition proteins appropriately to position their active sites outward from the gold surface to provide optimal interaction with target or substrate molecules. This is accomplished by placing the GBP domain at the N-, or C-termini, or within a surface loop of the recognition protein as indicated with linkers consisting of flexible amino acid sequences between domains. Conventional chemical attachments to GBP (Woodbury, et al., Sensors & Bioelectronics, 13:1117-1126, 1998) or other layers typically do not produce proper orientation to permit complete accessibility to binding sites on recognition proteins.

The expression plasmids disclosed in the present invention can be readily adapted for the production of virtually any polypeptide with just a few days effort. Once the expression hosts are created, unlimited quantities of many different GBP-containing recombinant proteins can be produced to create, for example, diverse arrays of proteins to facilitate proteomic research and drug screening. The gold-binding process is facilitated by the GBP domain common to each recombinant protein, thereby, ensuring attachment of all desired polypeptides, regardless of intrinsic, or lack of, attraction of the fusion partner to gold. Further, the one-to one relationship of GBP and its fusion partner allows the attachment to gold of equimolar amounts of hundreds or thousands of distinct recombinant molecules with different binding or enzyme activities. These benefits derived from the invention, herein, will significantly enhance the construction and performance of protein arrays, nanotechnology-based devices and the like.

The molecular approach described, herein, provides methods for introducing significant improvements in introducing a variety of functions to gold surfaces not possible by existing technology. For example, genetic engineering can produce a recombinant molecule containing GBP and the smallest possible form of a recognition protein that retains binding specificity. This provides at least three benefits. First, reduction of a protein to its specific binding domain eliminates other domains that may contribute complicating allosteric binding events or that could add to background interference. Second, in general, small functioning proteins are less susceptible than larger ones to proteolytic degradation when exposed to biologic fluids. Third, in the example of certain biosensing instruments, binding events occurring nearer the sensing surface produce stronger signals than those occurring farther away from the surface. Thus, the smaller the recognition protein, the higher the sensitivity of detection. A further benefit of the molecular approach is that appropriate modifications can be introduced into the protein sequence to produce a recombinant molecule with increased stability or other improvements. For example, if a region of the recombinant molecule is susceptible to proteolysis, introducing appropriate amino acid substitutions in the fusion protein may prevent degradation.

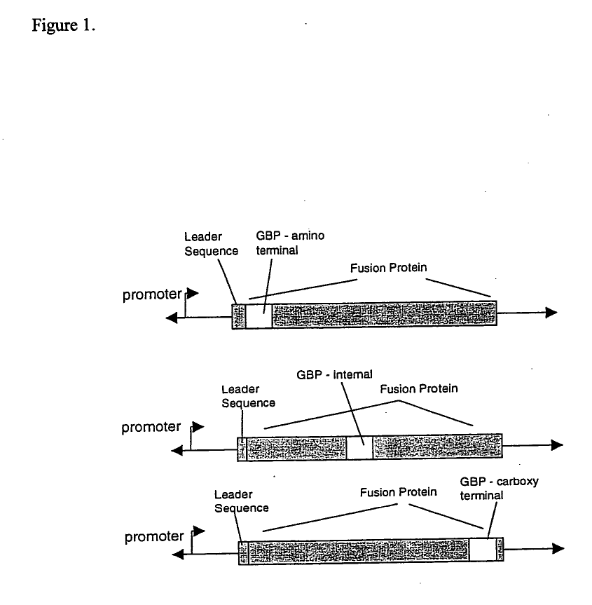

GBP fusion proteins can be arranged in several different ways as depicted in FIG. 1. The GBP sequence can be positioned at the amino terminus, internally or at the carboxyl terminus. The drawings represent the DNA sequence encoding the fusion protein portion of plasmid vectors that are expressed in bacterial, baculoviral, yeast, plant or mammalian cell hosts. It is apparent from the middle representation in FIG. 1 of an internally positioned GBP domain that two functional fusion partners, either identical partners or distinct partners can be placed in a single fusion protein. This novel feature of the inventive conception will be described in detail in specific examples below.

In this disclosure, we describe detailed methods for expressing GBP-based fusion proteins, rapid purification, characterization of activities, and provide specific examples for applications. Recognition proteins include, but are not limited to, protein A or G or related molecules, streptavidin or avidin or related molecules, single-chain antibodies, receptors, ligands, proteases, protease inhibitors, enzymes, enzyme inhibitors or any protein that specifically binds small molecules, cofactors or macromolecules. The latter group includes homo- or heterodimers or higher complexes of proteins and macromolecules required for a specific biologic function.

A patent has been issued for a “Method of producing IGG-binding protein as fusion peptides and a vector therefor” that utilizes protein A (Lofdahl, et al., U.S. Pat. No. 5,100,788). Another patent has been issued for a process to produce fusion proteins containing streptavidin (Cantor et al., U.S. Pat. No. 4,839,293). The disclosures and concepts of the present invention are beyond the scope of U.S. Pat. Nos. 5,100,788 and 4,839,293 and the applications provided herein were not disclosed or anticipated.

GBP-alkaline phosphatase chimera has been produced (Brown, Nat. Biotechnol. 15:269-272, 1997). The enzyme was fused to GBP solely as a reporter. Brown speculated that hybrid molecules containing metal-adhering peptides could bind to metallic sensor surfaces to provide more efficient procedures than are currently available. However, Brown does not disclose what these efficiencies are. Nor does Brown disclose how one reasonably skilled in the art can express and purify adequate amounts of stable hybrid molecules for commercial applications. The bacterial periplasmic expression system described by Brown produces only small quantities of GBP-alkaline phosphatase. Further, the expression of this particular fusion molecule may be preferentially favored because alkaline phosphatase is a normal periplasmic constituent. Many desired GBP-fusion proteins with commercial value may not be produced using Brown's expression system. Brown does not disclose alternative expression systems that those skilled in the art can use as a general strategy for the production of many different stable and active GBP-fusion proteins as described in the present invention. The prior art does not teach how stable GBP-fusion proteins can be expressed and purified in active form in large quantities as needed for commercial applications. Indeed, the prior art teaches that the expression and purification of each desired recombinant protein in active form are problematic. Brown does not disclose how those skilled in the art can overcome the unique set of difficulties encountered in the expression and purification of individual GBP-fusion proteins.

In addition, Brown does not disclose specific GBP-fusion proteins of commercial value that those skilled in the art can produce. For examples, Brown does not disclose the production of Streptavidin/Avidin-GBP, protein A/G-GBP, or single-chain antibody-GBP, or glucose oxidase-GBP, or horseradish peroxidase-GBP fusion proteins as the invention, herein, discloses and provides detailed examples for. Nor does Brown propose specific commercial applications of GBP-fusion proteins, beyond referring to the non-specific word “sensors”, in contrast to the examples of commercial application provided by the present invention. Finally, Brown does not disclose how GBP-fusion proteins can be used to fabricate functioning sensors. Prior art teaches that the construction of sensors coupled with the development of essential, rigorous assays can be extremely difficult endeavors without detailed instructions.

Woodbury and coworkers (Woodbury, et al., Sensors & Bioelectronics, 13:1117-1126, 1998; and U.S. Pat. No. 6,239,255) disclose a method for the chemical attachment of molecules to a GBP foundation layer on gold. Their methods are limited to the construction of biosensing instruments based on the optical principle of surface plasmon resonance. No disclosures or claims are made for the expression, purification, and applications of recombinant GBP-fusion proteins as conceived in the present invention.

We disclose, herein, detailed instructions for constructing unique expression vectors, for the production of large quantities of stable fusion proteins, for the determination of the activities of all fusion partners, and specific commercial applications for GBP-fusion proteins. The invention further discloses general expression and purification procedures capable of producing large quantities of stable, active fusion proteins with little effort and cost, thereby increasing the prospect of developing commercial applications.

### EXAMPLE 1

### Plasmid Design for Expression of GBP Fusion Proteins

Recombinant fusion proteins are produced by expression of plasmid constructs encoding the protein of interest fused with the GBP. The plasmid constructs include a selectable marker including but not limited to ampicillin resistance, kanamycin resistance, neomycin resistance or other selectable markers. Transcription of the GBP fusion protein is driven by a regulatable promoter specific for expression in bacteria, yeast, insect cells or mammalian cells. The construct includes a leader sequence for expression in the periplasmic space, for secretion in the media, or for expression in inclusion bodies in bacterial cells, or for secretion in yeast or mammalian cells. Plasmid constructs include multiple cloning sites for insertion of protein sequences in frame with respect to the GBP polypeptide. The GBP sequence can be inserted at the amino-terminal or C-terminal end of fusion partners or inserted within the coding sequence of the fusion partner. More than one GBP domain can be fused to a single fusion partner. More than one fusion partner can be fused to a single GBP sequence.

Here we describe the design of a modular set of vectors to support the production of amino and carboxyl terminal fusion proteins in E. coli expression systems. We included the addition of amino or carboxy affinity tags for purification; the addition of flexible linking sequences between domains to provide independent activity of fusion partners; the presence of a specific cleavage site to disconnect fusion partners if desired; and the requirement for highly regulated expression where toxicity of the over-expressed fusion protein could limit production.

General Methods:

Media. Strains and Transformation: LB media (Bacto L B broth, Miller, from Difco) was used as the basic growth media throughout the course of this study. The antibiotic ampicillin was used at a concentration of 150 μg/ml on plates and at 100 μg/ml in liquid media for the selection and growth of plasmid containing cells. NovaBlue cells from Novagen served as the E. coli host for transformation and expression. Transformations were performed according to the manufacturer's protocol.

Molecular Biology Supplies: All restriction endonucleases and T4 DNA ligase were purchased from New England Biolabs and the kit for DNA sequencing for the Big Dye terminator cycle sequencing from PE/ABI. Plasmid DNAs were made using the miniprep plasmid kits from Qiagen and DNA was extracted from agarose gel slices with is gel extraction kits from either Qiagen or Eppendorf. All reagents were used according to the manufactures' protocols.

Construction of the expression plasmid for Protein A-GBP fusion protein. The plasmid pSB3053 obtained from S. Brown (Brown, Nat. Biotechnol. 15:269-272, 1997) was used as the source of the GBP fragment containing seven repeats of the peptide MHGKTQATSGTIQS. Upon DNA sequencing it was found that the last repeat carried a substitution of the threonine residue in the fifth position for an isoleucine. All the fusion proteins constructed in this work have this substitution.

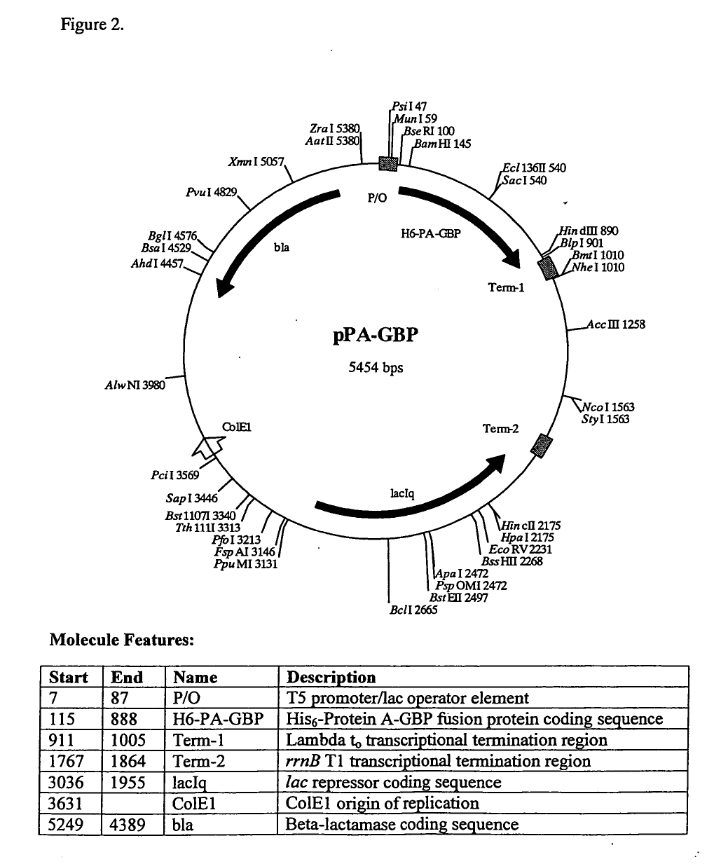

An EcoR I-Xho I fragment encompassing the GBP coding sequence was excised from pSB3053 and adapted at the 3′ end to include coding triplets for the amino acids EGP and a stop codon. Oligonucleotides BH3 (5′ TCG AGG GTC CGT AAT A 3′) and BH4 (5′ AGC TTA TTA CGG ACC C 3′) were annealed to obtain an adaptor with Xho I and Hind III cohesive ends. The EcoRI-Xho I GBP containing fragment and the adaptor were assembled in pUC18 and cut with EcoR I and Hind III in a three-part ligation to obtain plasmid pBHI-1. The Bsl I-Hind III fragment from pBHI-1 carrying the GBP coding sequence was adapted at its 5′ end to include an in-frame linker sequence with an Asn-Gly hydroxylamine sensitive cleavage site. Oligonucleotides BH1 (5′ CTG GTA GTG GCA ATG GTC ATA TGC 3′) and BH2 (5′ TAT GAC CAT TGC CAC TAC CAG AGC T 3′) were annealed to obtain an adaptor with Sac I and Bsl I cohesive ends. The adaptor also incorporates an Nde I site at the methionine codon of the first GBP repeat for ease of adaptation of the GBP fragment with any desired in-frame sequence. Plasmid pBHI-2 was generated with the Bsl I GBP fragment this adaptor and pUC19 linearized with Sac I and Hind III, in a three-part ligation. The nucleotide sequence of the Sac I-Hind III, double-adapted GBP fragment was confirmed by DNA sequencing. The Sac I-Hind III fragment from pBHI-2 was cloned between the Sac I and Hind III sites of pEZZ18 (Amersham) for an in-frame fusion with the two Z domains of staphylococcal Protein A (Nilsson, et al., Protein Eng 1:107-113, 1987) to obtain plasmid pBHI-3. The final expression plasmid for the cytoplasmic production of the His-tagged fusion protein was constructed by ligating the Protein A-GBP containing Fsp I-Hind III fragment from pBHI-3 and a short adaptor sequence formed by oligonucleotides BH11 and BH12 (5′ GAT CCG GTT CTG GTG C₃′ and 5′ GCA CCA GAA CCG 3′, respectively) into pQE-80L (Qiagen, Inc) cut with BamH I and Hind III. The resulting plasmid, called pPA-GBP, is depicted in FIG. 2. The nucleotide sequence of the encoded fusion protein was confirmed by DNA sequencing. The complete DNA sequence of pPA-GBP and the amino acid sequence of the fusion protein appear in the Sequence Listing section at the end of this document.

Construction of the expression plasmid for Streptavidin-GBP fusion protein. The coding sequence for core streptavidin residues 13-139 of the mature polypeptide (Sano, et al., J Biol Chem 270:28204-28209, 1995) was derived from a pUC18-based plasmid obtained from Dr. P. Stayton (Chilkoti et al., Proc Natl Acad Sci USA 92:1754-1758, 1995). A Sac I restriction site was engineered into the coding sequence to allow fusions to the shortened version of streptavidin, residues 13-133 (Sano, et al., J Biol Chem 270:28204-28209, 1995). For this, an EcoR 1-Mlu I fragment encoding the partial core streptavidin sequence was linked to an adaptor with Mlu I and Hind III cohesive ends (formed using oligo pairs BH7/BH8, 5′ CGC GTG GAA ATC CAC CCT GGT TGG TCA 3′/5′ GTG TCG TGA CCA ACC AGG GTG GAT TTC CA 3′ and BH9/BH10 5′ CGA CAC CTT CAC CAA AGT TTC GAG CTC 3′/5′ AGC TTG AGC TCG AAA CTT TGG TGA AG 3′) and inserted into pUC18 cut with EcoR I and Hind III to yield pBHI-5. The nucleotide sequence of the total EcoR I-Hind III insert in pBHI-5 was confirmed by DNA sequencing.

Using an Nde I site present at the initiating methionine of the adapted core streptavidin sequence in pBHI-5, the Nde I-Hind III fragment encoding core streptavidin was cloned into the expression vector pQE-80L (Qiagen, Inc), digested with BamH I and Hind III. A short adaptor sequence with BamH I and Nde I cohesive ends, formed with the oligo pair BH17/BH18 (5′ GAT CCG GTT CTG GTG GCC A 3′/5′ TAT GGC CAC CAG AAC CG 3′) was used for linking.

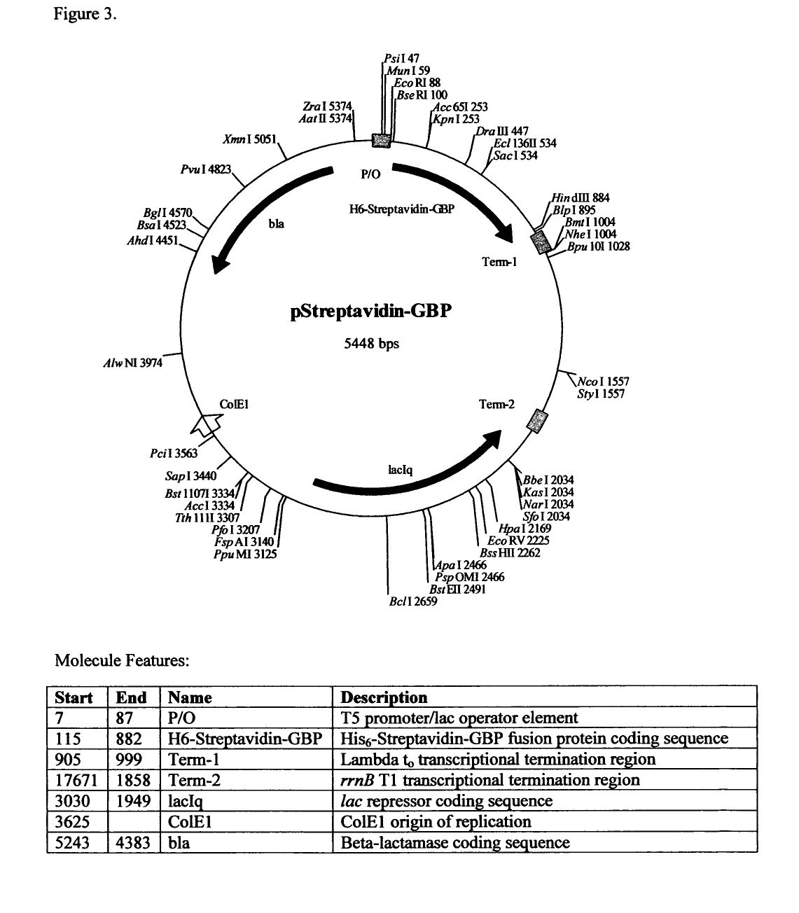

The resulting plasmid called pBHI-7 can produce a N-terminal His-tagged core streptavidin molecule residues 13-133, ending with the added amino acid residues SSSSILS. To express the His-tagged core streptavidin-GBP fusion protein, the engineered Sac I site in the core streptavidin sequence (see above) was utilized to link the Sac I-Hind III GBP encoding fragment from pBHI-2 to generate the expression plasmid pStreptavidin-GBP which has the basic backbone of the expression vector pQE 80L (Qiagen, Inc). The plasmid map, pStreptavidin-GBP is depicted in FIG. 3 and relevant DNA and amino acid sequences appear in the Sequence Listing section at the end of this document.

In summary, we have produced vectors for the expression of His₆-protein A-GBP, His₆-streptavidin-GBP and His₆-streptavidin lacking the GBP as a control protein. In addition we have subcloned the GBP as a modular cassette to support the development of future recombinant fusion proteins.

The expression constructs contain DNA that encodes repeating glycyl-seryl sequences to provide flexible linkers between domains for maximizing independent activities of domains.

The expression constructs contain DNA that encodes specific chemical cleavage sites including, but not limited to, asparaginyl-glycyl or aspartyl-prolyl bonds (Bornstein and Balian, Methods Enzymol 47:132-145, 1977; Szoka, et al., DNA 5:11-20, 1986). The invention also provides for DNA that encodes specific protease cleavage sequences for Factor Xₐ or Enterokinase and the like (Jenny, et al., Protein Expr Purif 31:1-11, 2003; Wang, et al., Biol Chem Hoppe Seyler 376:681-684, 1995).

The expression constructs contain DNA that encodes an affinity “tag” sequence, for example, but not limited to, polyhistidine, V-5 epitope, or FLAG epitope to facilitate rapid, one-step purification of fusion proteins (Dobeli, et al., U.S. Pat. No. 5,047,513; Chen, et al., Eur J Biochem 214:845-852, 1993; Terpe, Appl Microbiol Biotechnol 60:523-533, 2003).

### EXAMPLE 2

### Expression of GBP-Fusion Proteins

The GBP-fusion constructs for all examples were transfected into NovaBlue cells (Novagen). For expression, an overnight culture of the transformants grown in LB broth+ampicillin at 37° C. was diluted into fresh media and grown with vigorous shaking till the OD measured at 600 nm was between of 0.3-0.4. Isopropyl β-D-thio-galactopyranoside was added to a final concentration of 4 mM and the incubation was continued for another 4 hours. The cells were collected by centrifugation, washed once with 150 mM KCl and frozen.

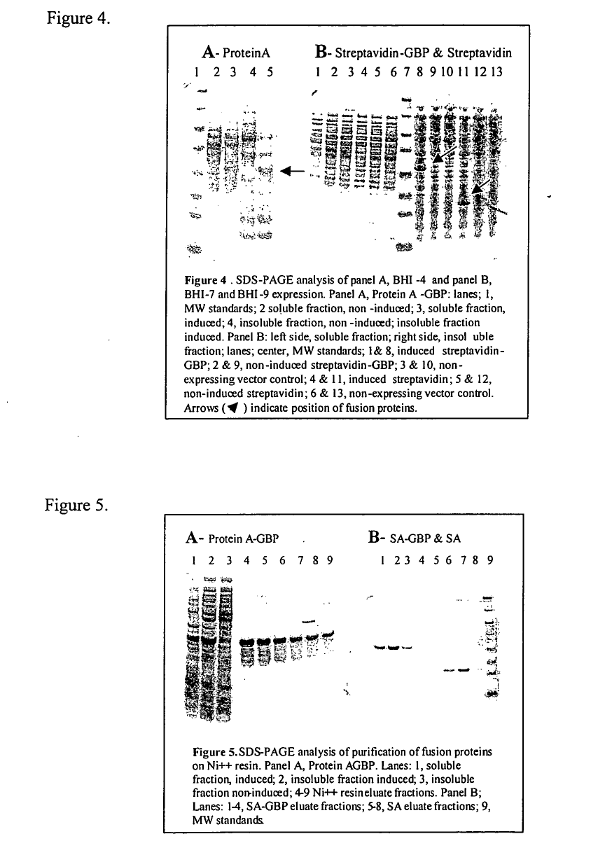

In preliminary experiments, induced and non-induced cells were first extracted in B-Per (Pierce), a gentle buffer for lysis of bacteria to recover soluble proteins. The extract was centrifuged to clarify the solution and the pellet was extracted directly in SDS-PAGE sample buffer to recover insoluble proteins. All samples were analyzed by SDS-PAGE and staining with a colloidal form of coomasie blue (Invitrogen). The results of these experiments shown in FIG. 4 indicate that high levels of His₆-protein A-GBP and His₆-streptavidin-GBP fusion proteins were produced by induced cells and little, if any, protein was observed in non-induced cells. Thus, our repressible/inducible system functioned as expected. Further, there was no apparent proteolytic degradation of the fusion protein during culture or the extraction procedure. In the case of His₆-protein A-GBP, some of the fusion protein appeared to be in the soluble fraction, but most was observed in the SDS-PAGE sample buffer extracts. In contrast, essentially all of the His₆-streptavidin-GBP fusion was insoluble and required SDS to extract the protein. A His₆-streptavidin construct lacking the GBP domain was also expressed and the resulting protein had solubility properties similar to those of the molecule containing GBP.

The fusion partners were observed to bind gold powder directly from the crude cellular extracts as evident by SDS-PAGE analysis of the gold powder. A few, very abundant E. coli proteins also bound gold but it was clear the GBP-fusions preferentially bound gold (data not shown).

### EXAMPLE 3

### Purification of GBP-Fusion Proteins

Larger cultures were grown to produce sufficient fusion proteins for purification and characterization. To extract proteins under “native” conditions for subsequent purification, the bacteria were resuspended in 50 mM sodium phosphate buffer, pH 8.0, containing 0.5M sodium chloride and 10 mM imidazole to a final density approximately 20 times greater than that of the original cultures. Cells on ice were lysed by sonication at medium power and interval setting of 50% to give an intermittent pulse for 30 seconds. This was repeated for 6 cycles with one-minute rest on ice between cycles. Following each cycle, the optical density at 600 nm was recorded to assess cell lyses. The sonicated suspension was centrifuged 5,000×g for 10 min to remove cell debris and insoluble proteins from the soluble fraction. The resulting pellet was extracted in a “denaturing” solution of 20 mM sodium phosphate buffer, pH 7.8, containing 6M guanidine HCl (Gu-HCl) and 0.5M sodium chloride and the suspension was centrifuged to remove insoluble material.

In the case of the streptavidin fusion proteins, the cells were extracted only with 20 mM sodium phosphate buffer, pH7.8, containing 6M Gu-HCl and 0.5M sodium chloride.

Purification of His₆-protein A-GBP, His₆-streptavidin-GBP, and His₆-streptavidin fusion proteins. The His₆-tag recombinant proteins, were purified on ProBond nickel-resin columns (Invitrogen) as recommended by the manufacturer. Material in the two extracts, i.e., under native conditions for soluble proteins or denaturing conditions for insoluble proteins, was incubated with individual Probond Nickel resin columns, washed, and eluted as recommended by the manufacturer. Analysis by SDS-PAGE shown in FIG. 5 indicated that the final preparations were 90%-95% pure accompanied by proteolysis of a small amount of material, probably at the GBP domain. Initial extracts did not include protease inhibitors, but future preparations will include PMSF and a commercial “cocktail” of protease inhibitors. The optical density at 280 nm of the eluate fractions was recorded and the peak fractions from each column were pooled, aliquoted and stored at −20° C. Interestingly, sonication solubilized at least 80% of the total His₆-protein A-GBP. Thus, one-step purification of stable recombinant His₆-protein A-GBP, His₆-streptavidin-GBP, and His₆-streptavidin proteins was possible in just a few hours from cell extraction to pure protein.

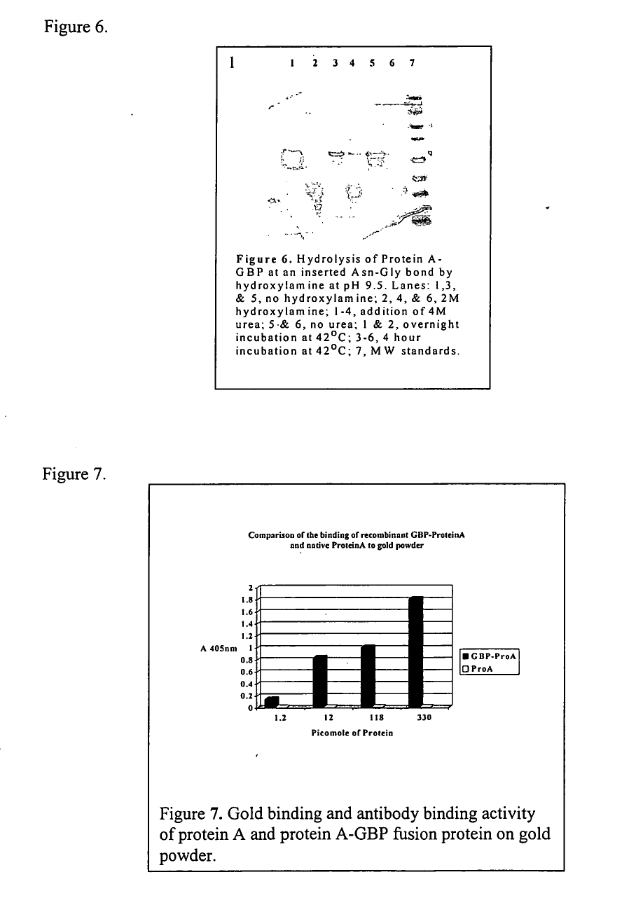

The inclusion of an Asn-Gly bond, susceptible to hydrolysis in 2M hydroxylamine and 4M urea at pH 9.5, allowed us to physically dissociate GBP from protein A as shown in FIG. 6. As a method to achieve limited digestion of proteins, urea is required to unfold proteins to make any Asn-Gly bonds fully accessible to hydroxylamine. However, because of the exposed location of our inserted Asn-Gly bond we achieved efficient hydrolysis without adding urea in just a few hours. Further, it was possible to hydrolyze the fusion protein while it was bound to gold powder (data not shown). Thus, it is highly probable that we can selectively hydrolyze fusion proteins at our inserted Asn-Gly site even when fusion partners contain such bonds, especially if even less stringent conditions can be employed. In proposed phase II research we will optimize the cleavage conditions and investigate the use of other specific sequences for restricted polypeptide cleavage including Asp-Pro bonds or by Factor Xa.

### EXAMPLE 4

### Characterization of GBP-Fusion Proteins

Colorimetric assays were developed to determine gold-binding activity of GBP and fusion partner activities of the purified recombinant proteins. Spherical gold powder (Sigma-Aldrich), 1.5 to 3 micron in size, was washed overnight at room temperature in hydrofluoric acid to remove contaminants (Brown, Nat. Biotechnol. 15:269-272, 1997). Samples containing 0 to 330 picomole of purified His₆-GBP-protein A or native protein A (Sigma) were diluted in 1 mL 10 mM potassium phosphate, pH 7.0, containing 100 mM potassium chloride and 1% triton X-100 (PKT buffer) and incubated in 2 mL centrifuge tubes with 1 mg of gold powder for 5 min at room temperature with gentle mixing. Samples containing 0 to 22 picomole of puriifed recombinant His₆-streptavidin-GBP or His₆-streptavidin were similarly prepared. Gold powder was collected by centrifugation at 10,000×g for 1 min and incubated in 1 mL of phosphate buffered saline (PBS), pH 7.4, containing 2 mg bovine serum albumin (BSA)/mL for 5 min with mixing. The gold powder was then rinsed twice in a 1:1 solution of PKT and PBS/BSA buffers.

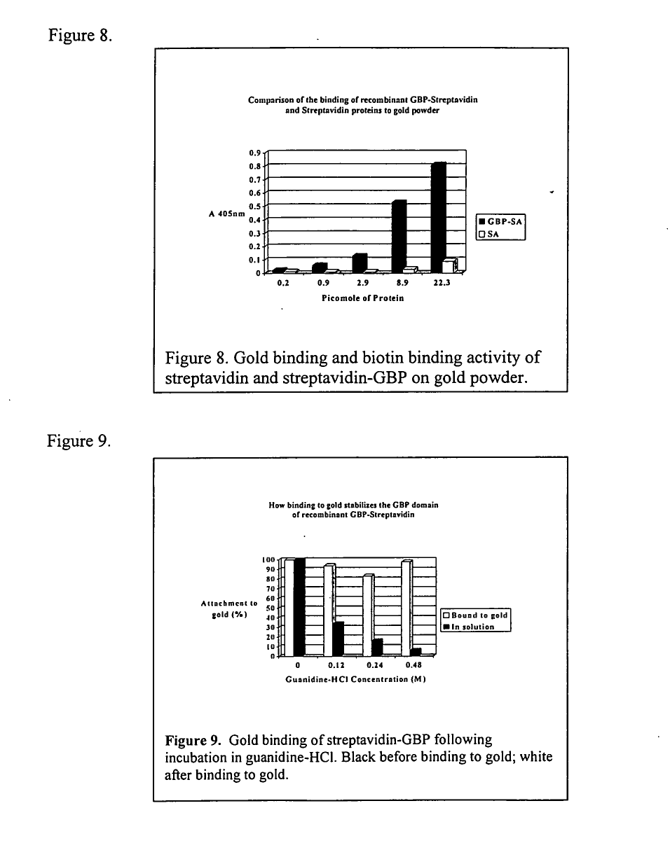

In the case of the protein A samples, mouse monoclonal IgG₁ antibody (anti-FLAG, Sigma-Aldrich) labeled with alkaline phosphatase was incubated at room temperature at a dilution of 1:1000 with the gold powder in 1 mL of a 1:1 solution of PKT and PBS/BSA buffers for 15 min with mixing. To assess streptavidin activity, biotinylated goat antiserum with specificity to mouse immunoglobulin (Sigma-Aldrich) was incubated at room temperature at a dilution of 1:1000 with the gold powder in 1 mL of a 1:1 solution of PKT and PBS/BSA buffers for 15 min with mixing. The gold powder was rinsed twice in 1 mL of 1:1 solution of PKT and PBS/BSA buffer, and incubated with 1 mL of a 1:1000 dilution of mouse monoclonal (anti-rabbit) conjugated alkaline phosphatase in a 1:1 solution of PKT and PBS/BSA buffers for 15 min with mixing. The gold powder was washed twice in 1 mL of 1:1 solution of PKT and PBS/BSA buffer, transferred to unused centrifuge tubes, and assayed for alkaline phosphatase activity in 1 mL of p-nitrophenylphosphate in 50 mM Tris-HCl, pH 8.0, (51 mg in 25 mL) at room temperature with mixing over time. The reaction was stopped by removing the gold by centrifugation. The optical densities at 405 nm of the supernatant fluids were recorded. The results shown in FIGS. 7 and 8 indicate that the recombinant proteins contain both functional GBP domain and fusion partner activities. Further, the results establish the remarkable ability of GBP to facilitate specific gold binding of proteins at very low concentrations compared to direct adsorption of protein A and His₆-streptavidin which, lacking the GBP domain, bind minimally to gold powder in PKT buffer.

The concentration range for the recombinant streptavidin proteins was less than that for protein A because these proteins were still in 6M Gu-HCl following purification and preliminary studies indicated that gold binding by His₆-streptavidin-GBP was inhibited at relatively low Gu-HCl concentrations. This was not unexpected because GBP contains no disulfide bonds to help stabilize the polypeptide's tertiary structure. Future studies will be performed in the absence of Gu-HCl to determine levels of protein needed to saturate gold, however, the observation of the effect of this agent on gold-binding was fortuitous. Additional studies were conducted to gain further insight regarding GBP gold binding properties. There is a possibility that inhibition of gold binding was not a direct effect of Gu-HCl on GBP, but rather the guanidinium ion could compete with GBP for binding sites on gold in PKT buffer. If so, the ions must bind tightly to gold to block GBP attachment. Therefore, samples of gold powder were washed with up to 0.5M Gu-HCl in PKT buffer, recovered by centrifugation prior to binding His₆-streptavidin-GBP in PKT buffer, and compared to the binding of fusion protein to gold not washed with Gu-HCl. The results (data not shown) indicated near identical His₆-streptavidin-GBP binding to gold powder whether or not the powder was pre-washed with 0.5M Gu-HCl suggesting that the original observation of Gu-HCl inhibition of fusion protein binding to gold was a direct effect of the agent on GBP.

This study was followed by one to assess the stability of His₆-streptavidin-GBP already attached to gold powder in the presence of PKT buffer containing increasing concentration of Gu-HCl. The results shown in FIG. 9 indicate that once formed the GBP/gold interaction is remarkably stable when exposed to a strong chaotropic agent such as Gu-HCl. Indeed, following incubation in 3M and 6M Gu-HCl, there was 70% and 30% retention of His₆-streptavidin-GBP binding to gold powder, respectively (data not shown). The observed stability for the GBP/gold interaction in this study is likely an underestimate since hydrofluoric acid-treated gold powder still contains contaminants that may preclude optimum interaction of some molecules of GBP with gold (Brown, Nat. Biotechnol. 15:269-272, 1997). Nevertheless, the results indicate that robust biosensors and other applications will be supported by these GBP-fusion proteins.

### EXAMPLE 5

### Construction and Characterization of Biosensors

Surface plasmon resonance (SPR)- an optical principle-biosensors were constructed on a fully integrated miniature SPR transducer, called Spreeta, from Texas Instruments (Melendez, et al., Sensors & Actuators B, 35, 36:212-216, 1996). Sensor chips were coated with recombinant His₆-protein A-GBP and His₆-streptavidin-GBP and the performance of each was compared to that of control sensors constructed with native protein A or recombinant streptavidin lacking the GBP domain. Solutions were delivered by a peristaltic pump at a flow rate of 0.2 mL/min at room temperature through a flow cell attached to each sensor. Clean sensing surfaces were rinsed initially for 10 min in 10 mM potassium phosphate buffer, pH 7.0 containing 10 mM potassium chloride and 1% Triton X-100 (PKT buffer) followed by solutions of PKT buffer containing test proteins. In the case of protein A-GBP or native protein A, the gold sensing surfaces were incubated for 10 min with 12 picomole of protein/mL For recombinant His₆-streptavidin-GBP or His₆-streptavidin 4.5 picomole of each /mL was used. Again, the presence of Gu-HCl precluded using higher amounts of protein. Future studies will use solutions without Gu-HCl, but in the current studies the concentration of proteins was sufficient to saturate the tiny sensing area. Following the application of protein, the sensors were rinsed with PKT buffer and then phosphate buffered saline, pH 7.4, containing 2 mg bovine serum albumin/mL (PBS/BSA buffer) for 10 minutes each. This completed the process to construct a sensor.

All antibodies were diluted at 1:1000 in PBS/BSA buffer for sensing evaluation. All solutions flowed over the sensing surface for 10 min each with the exception of 20 min for 0.1M glycine-HCl, pH 2.0, used to regenerate the surface. Refractive index (RI) vs. time was recorded by Spreeta software on a laptop commuter.

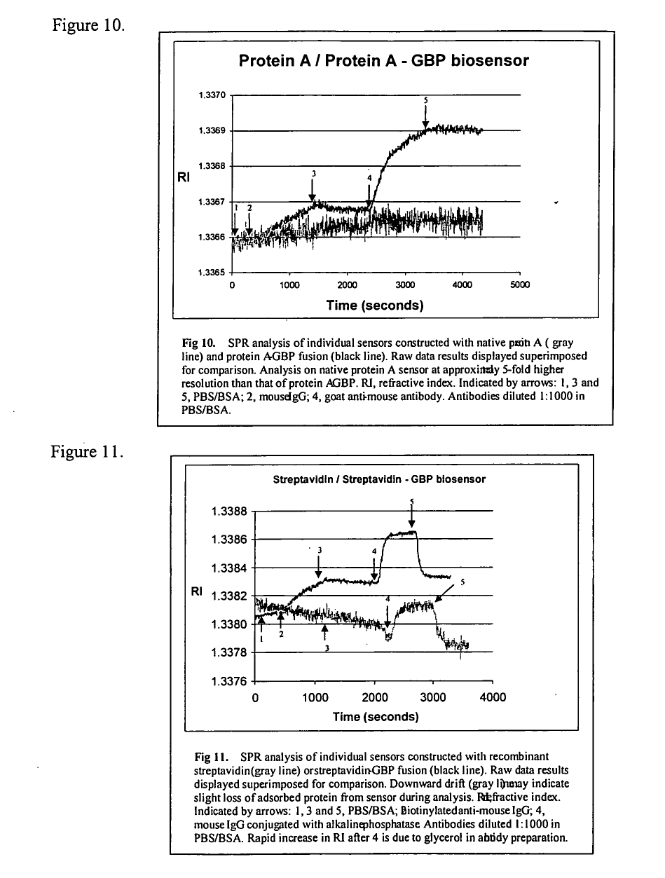

a) Recombinant His₆-protein A-GBP and native protein A. To evaluate their performance each sensor was exposed to mouse monoclonal IgG (anti-FLAG), rinsed in PBS/BSA buffer, exposed to polyclonal goat anti-mouse, and rinsed in PBS/BSA buffer. This procedure effectively eliminates non-specific antibody binding. The results shown in FIG. 10 indicate excellent gold- and immunoglobulin-binding activities for His₆-protein A-GBP as anticipated from the results of studies with gold powder. Also, as expected, there was no evidence of binding with native protein A. Exposure of the His₆-protein A-GBP based sensor to 0.1M glycine-HCl, pH 2.0, regenerated the sensing surface and allowed a second high-quality analysis (data not shown). No evidence of sensing fouling in the presence of BSA or antibodies was observed.

b. Recombinant His₆-streptavidin-GBP and His₆-streptavidin: Each sensor was exposed to biotinylated goat anti-mouse antibody, rinsed in PBS/BSA, buffer, exposed to mouse IgG (conjugated with alkaline phosphatase), and rinsed in PBS/BSA buffer. The results shown in FIG. 11 indicate that a very robust sensor was constructed with His₆-streptavidin-GBP, but not with His₆-streptavidin lacking GBP. The rapid increase in RI when mouse IgG was introduced was due to glycerol in the stock preparation. The signal for capturing mouse IgG by anti-mouse antibody held firm to His₆-streptavidin-GBP was significant, but less than expected probably because some of the epitopes on the conjugated target were blocked. As with a) above the sensor was regenerated by removing the mouse IgG in 0.1M glycine-HCl, pH 2.0, allowing a second analysis for capture of mouse IgG. There was no evidence of sensor fouling by BSA or antibodies.

The sensor constructed with His₆-streptavidin without GBP completely lacked activity. While the protein was applied to the sensor it was evident that material bound initially to the sensing surface, but was partially washed off during the extensive rinse step. Also, during the PBS/BSA rinse, BSA evidently bound to the sensor displacing the remaining His₆-streptavidin; an observation not observed when applying GBP-fusion proteins (data not shown). The rinse steps here were much more extensive than those for the gold powder assays that indicated very low, but detectable streptavidin binding. Thus, under the conditions used, streptavidin lacking GBP is rather loosely adsorbed to gold surfaces whereas GBP-mediated binding is extremely stable.

The different response to glycerol in FIG. 11 is due to differences in individual sensor operation. Also, the downward drift of the signal for streptavidin lacking GBP may be due to loss of small amounts of protein from the surface during analysis.

### EXAMPLE 6

### Relative Specific Activity of Proteins on Gold Powder

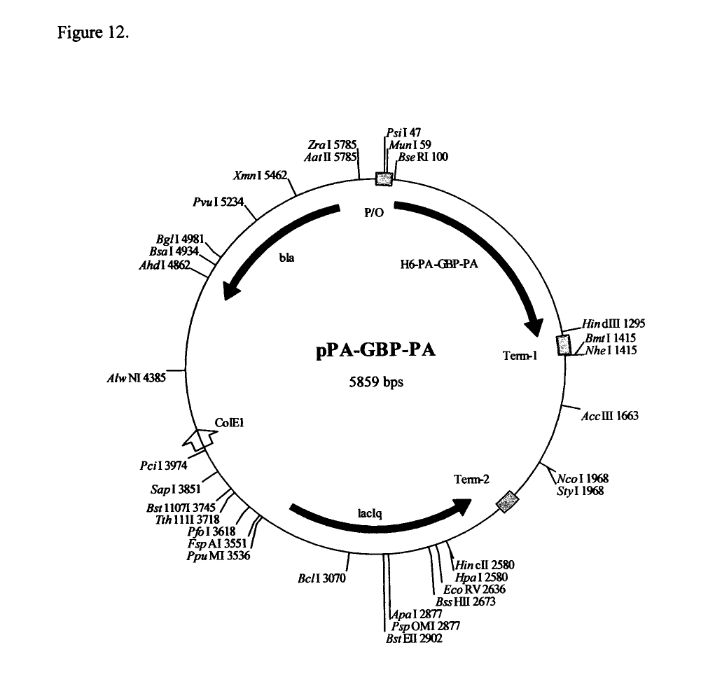

His₆-streptavidin-GBP attached to gold powder bound 5-10 fold more biotinylated antibody than a similar amount of the recombinant His₆-streptavidin based on SDS-PAGE analysis of the extracted protein. The low activity of His₆-streptavidin on gold (see FIGS. 9 and 12) is not a true indication of how much of this protein was adsorbed to gold. The preliminary studies did not carefully quantify the protein concentration. However, the implication is that properly oriented His₆-streptavidin-GBP on gold is much more effective in binding biotinylated molecules than is physically adsorbed His₆-streptavidin. This observation which was not predicted by the prior art indicates that the controlled orientation of GBP-fusion proteins on gold surfaces presents completely accessible binding/active sites resulting in many times more activity than that achieved by physical adsorption or conventional protein chemistry. This is an important benefit achieved by the present invention.

### EXAMPLE 7

### Production of Recombinant Proteins Consisting of a Single Domain of GBP and Multiple Copies of an Individual Fusion Partner

The results presented, herein, in Examples 1 through 6, establish that GBP-fusions can be expressed as stable proteins and rapidly purified with retention of gold-binding and other functions when fusion partners are attached to its amino-terminus of GBP. With the observation that a fusion partner also can be attached to the carboxy-terminus of GBP (Brown, Nat. Biotechnol. 15:269-272, 1997), our discoveries establish that GBP can accommodate fusion partners at either end of the polypeptide sequence. Consequently, the expression vectors described in Example 1 and depicted in FIG. 1 of this invention can be used to encode a recombinant protein containing a single GBP domain and a minimum of two identical copies of a specific polypeptide fusion partner. Without limiting the scope of the current invention, the following constructions are given as examples:

In one embodiment, a His₆-protein A-GBP-protein A fusion protein has been expressed in E. coli using the plasmid, pPA-GBP-PA, depicted in FIG. 12, and purified using the His₆ affinity tag.

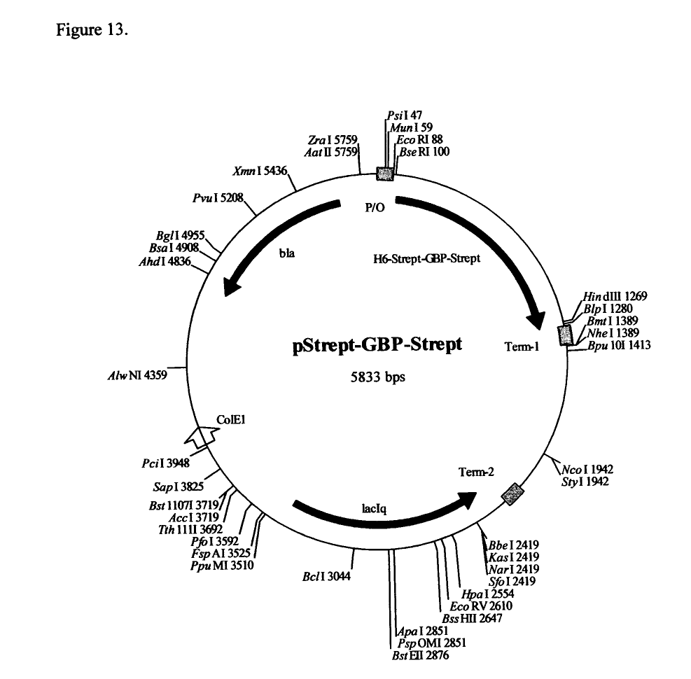

In another embodiment, a His₆-streptavidin-GBP-streptavidin fusion protein has been expressed in E coli using the plasmid, pStrept-GBP-Strept, depicted in FIG. 13, and purified using the His₆ affinity tag.

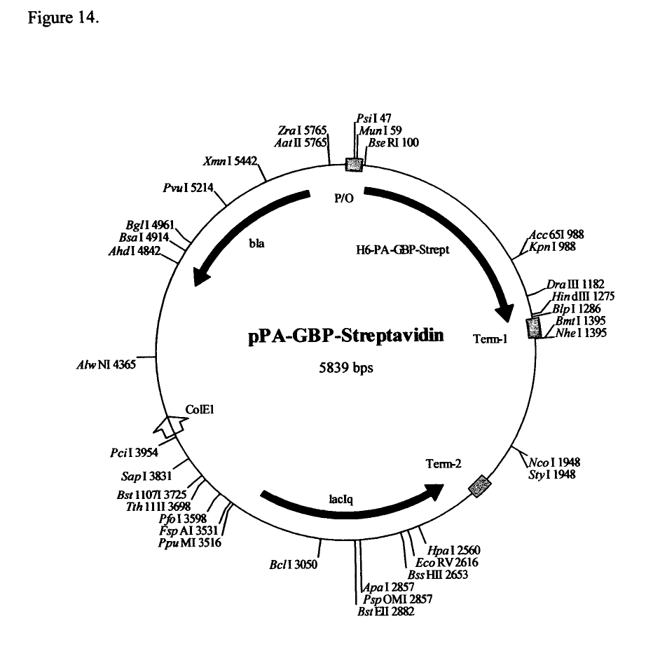

In another embodiment, a His₆-GBP fusion protein has been expressed in E coli using the plasmid, pGBP, depicted in FIG. 14, and purified using the His₆ affinity tag.

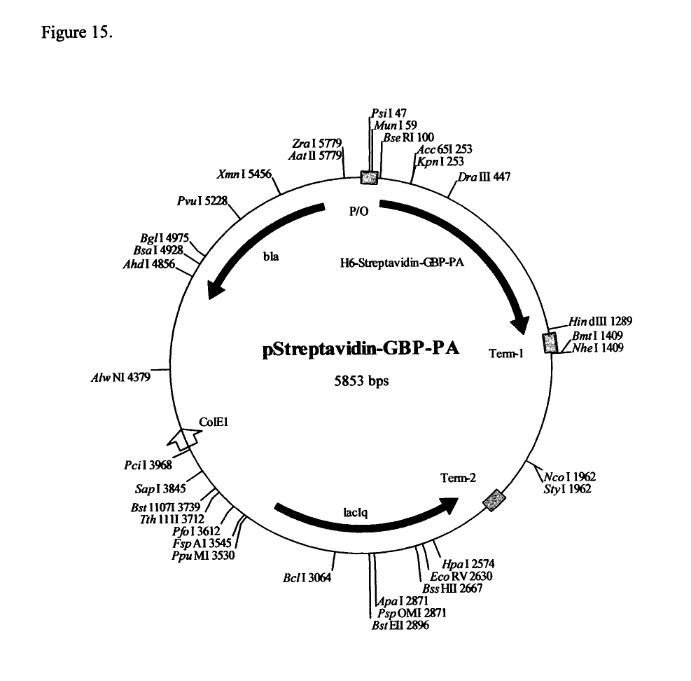

In another embodiment, a His₆-GBP-GBP fusion protein has been expressed in E coli using the plasmid, pGBP-GBP, depicted in FIG. 15, and purified using the His₆ affinity tag.

In other embodiments where physically and chemically permitted, the invention allows as instructed by Example 1 above more than one copy of fusion partner linked in tandem at either or both ends of GBP. The presence of flexible linking sequences consisting of glycyl-seryl repeats in the fusion proteins, allows for independent function of each domain of the fusion protein.

Without limiting the scope of the current invention, an example of how multiple copies of a specific fusion partner coupled to GBP can be advantageous relates to the field of biosensors. Biosensors, in general, perform at greater sensitivity with increasing density of recognition molecules, e.g., specific antibody, at the sensing surface. In the specific case of surface plasmon resonance (SPR)-based sensors, the ability to directly detect small analytes in real-time depends on the number of resonance units (RU) that are directly proportional to the density of analyte binding sites at the sensing surface. Similar increases in sensitivity and enhanced performance as illustrated in Example 6 above can be achieved for applications in all fields utilizing gold. Thus, the current invention provides important advantages in overall application performance not provided by existing methods, e.g., random physical adsorption of protein to gold or chemical attachment to foundation layers on gold.

There can be utility in using the recombinant His₆-GBP and His₆-GBP-GBP as agents to block the binding to gold of non targeted substances in test samples following any method to derivitize a gold surface.

### EXAMPLE 8

### Production of Recombinant Proteins Consisting of a Single GBP Domain and at Least One Domain Each of Two Different Fusion Partners

GBP-fusion proteins containing two distinct fusion partners with different function can have broad utility in all fields utilizing gold.

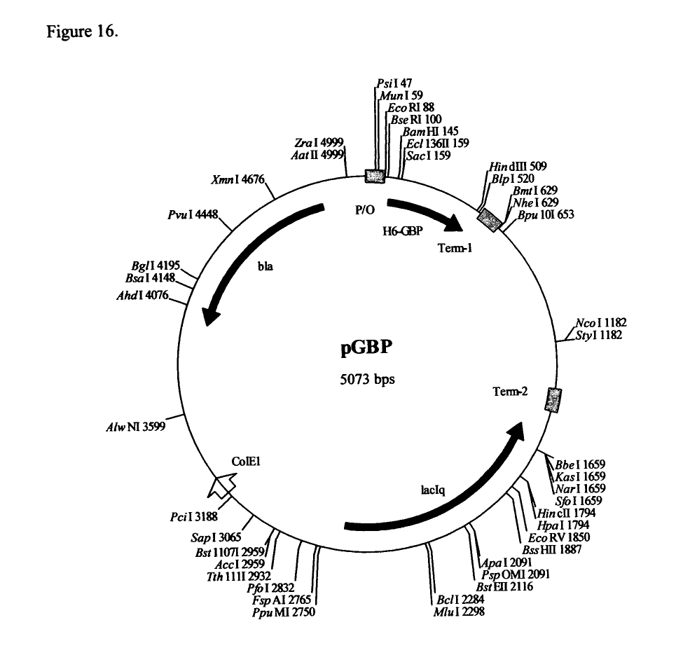

In one embodiment, His₆-protein A-GBP-streptavidin fusion protein has been expressed in E. coli using plasmid, pPA-GBP-Streptavidin, as depicted in FIG. 16, and purified using the His₆ affinity tag.

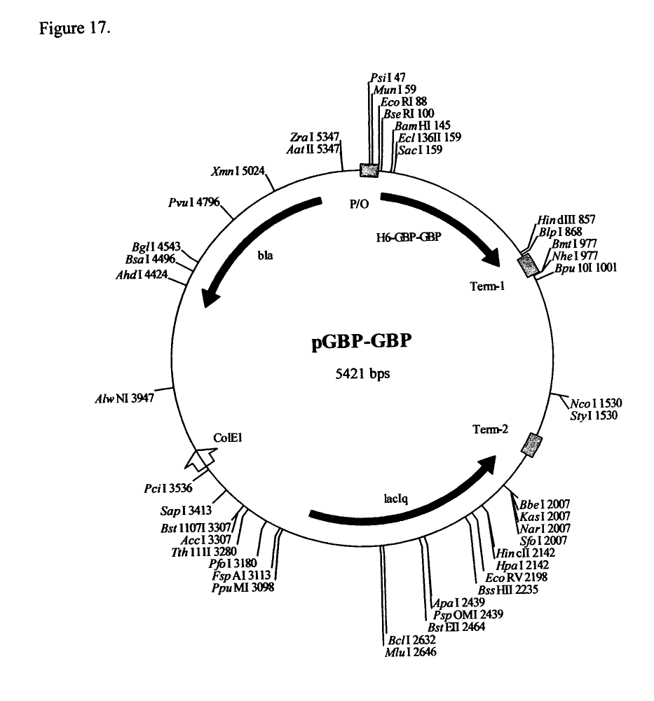

In another embodiment, His₆-streptavidin-GBP-protein A fusion protein has been expressed in E. coli using plasmid, pStreptavidin-GBP-PA, as depicted in FIG. 17, and purified using the His₆ affinity tag.

In another embodiment, GBP-fusion partners can be Protein A and other related polypeptides such as Protein G or Protein L or other similar proteins that have immunoglobulin-binding properties distinct from those of Protein A. Such a fusion protein provides the benefit of allowing the detection and binding of more than one class of immunoglobulin simultaneously or sequentially.

In another embodiment, the different GBP-fusion partners can be any two polypeptides with distinct affinity binding activity.

One advantage of fusion proteins with mixed function as described is to provide versatility by allowing, for example in the case of protein A-GBP-streptavidin, antibody binding activity and any other activity conferred by attachment of biotinylated-molecules, used either sequentially or concurrently. Versatile sensing chips and other surfaces can be constructed using these unique reagents to introduce multiple activities and to achieve improved efficiency and cost reduction compared to the use of existing reagents.

### EXAMPLE 9

### Production of Recombinant Proteins Consisting of Single-Chain Antibodies Fused to GBP

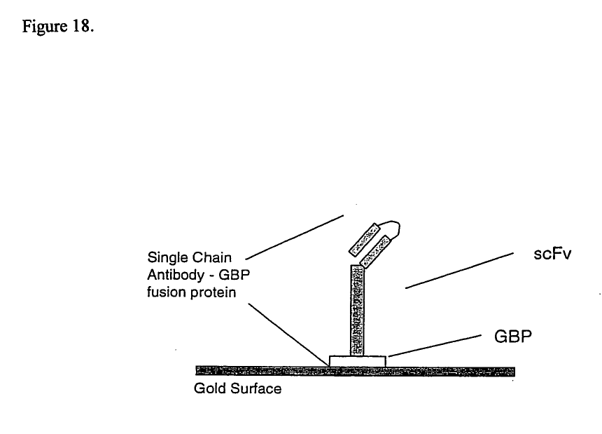

Single chain antibodies (scFvs) consist of variable domains (Fv) separated by linker sequences. Fusion of the scFv construct with different sequences encoding different function has been described. Carboxyl terminal fusion with the gene encoding stretpavidin produces an active scFv:streptavidin fusion protein (Kipriyanov, et al., Hum Antibodies Hybridomas 6:93-101, 1995). Cloning of the GBP sequence at the carboxyl terminus of scFv gene sequences produces scFv:GBP fusion constructs which can be expressed in bacteria as described in Example 1 above. Recombinant single-chain antibody fusions produced in this manner can be used to functionalize gold surfaces as illustrated in FIG. 18.

DNA sequences encoding specific single chain antibodies can be obtained by phage selection methods (Clackson, et al., Nature 352:624-628, 1991) or from hybridomas producing monoclonal antibodies. Using our expression plasmids described above in Example 1, those skilled in the art can link the GBP encoding sequence at the C-terminus, or where necessary, at the N-terminus of the sequence encoding scFv antibody. The fusion protein can be expressed, but not limited to, in the cytoplasm of E. coli NovaBlue cells (Novagen) with a His₆-tag at the N-terminus using the QE-80L series of expression vectors (Qiagen). The fusion proteins are likely to accumulate in inclusion bodies and can be purified using a Ni++ column and refolded (Huston, et al., Proc. Natl. Acad. Sci. USA 85:5879-5883, 1988). If necessary, an immuno-affinity purification step can be used to separate out the inactive molecules. If the product is not active, the domains can be shuffled around or periplasmic expression can be employed.

In one embodiment, a GBP-fusion protein can have a scFv fusion partner that has specificity for Clostridium botulinum toxin A.

In another embodiment, GBP-fusion proteins can have scFv fusion partners that in combination have specificity for six other serotypes of Clostridium botulinum toxin.

In another embodiment, a GBP-fusion protein can have a scFv fusion partner that has specificity for the toxin, ricin.

In another embodiment, a GBP-fusion protein can have a scFv fusion partner that has specificity for enterotoxin B from Staphylococcus aureus.

In another embodiment, GBP-fusion proteins can have scFv fusion partners that have specificity for of the Category A-D list of toxins and agents for biowarfare.

In other embodiments, GBP-fusion proteins can have scFv fusion partners with specificity to any toxin or poisonous agent.

In another embodiment, a GBP-fusion protein can have a scFv fusion partner that has specificity for anthrax spores.

In other embodiments, GBP-fusion proteins can have scFv fusion partners with specificity to any infectious agent.

In other embodiments, GBP-fusion proteins can have scFv fusion partners with specificity to important clinical targets, e.g. to the drug digoxin.

In other embodiments, GBP-fusion proteins can have scFv fusion partners with specificity to important environmental targets.

In other embodiments, two identical copies of a specific scFv can be fused to a single domain of GBP to provide increased analyte-binding capacity to a given area of gold surface. This can increase the sensitivity of the signal out-put of biodetection instruments during testing.

In another embodiment, the different GBP-fusion partners can be scFv antibodies with distinct specificity such as scFv1-GBP-scFv2. One advantage of such a fusion protein is to provide a means to concentrate two distinct molecules with interactive or reactive potential. This can be especially beneficial in cases where the dilute concentrations of two or more molecules in solution preclude their interactive or reactive potential.

### EXAMPLE 10

### Production of Recombinant Proteins Consisting of an Enzyme, Horse Radish Peroxidase, Fused to GBP

The production of fusion proteins containing certain enzymes and GBP provides a method to bind enzymes to gold with retention of optimal enzyme activity and other properties as generally described in this invention for any protein of interest. Currently, certain enzymes support applications in clinical testing, research, and industry generating total annual revenues of billions of dollars. These rapidly growing markets include glucose monitoring for diabetics, $3 billion/year; industrial enzyme use, $2 billion/year; and hundreds of millions of dollars annually for research enzymes. New monitoring devices intended for home use now under development, e.g., for cholesterol testing, will generate even larger markets.

The trends to smaller (nanotechnology), less expensive testing devices for home monitoring and research instruments requires innovative solutions to improve the efficiency of enzyme-based and other types of assays to support these devices. In particular, there is great demand for non-invasive or minimally invasive monitoring procedures. For example, if testing sensitivity can be increased above that of existing devices, many clinical tests can be developed to test salvia rather than blood. Also, the availability of more sensitive, low cost testing devices will facilitate the development of new monitoring approaches designed for home management of chronic diseases where daily testing would be beneficial. The invention disclosed, herein, will facilitate the fabrication of nanodevices in many fields because of several efficiencies (as previously described) that our novel technology of controlled orientation attachment of protein provides compared to existing methods.

The examples provided below for HRP or related peroxidase enzymes are intended to show the advantages and benefits of the use of enzyme-GBP fusion proteins compared to existing methods, without limiting the scope of the invention.

In one embodiment, the invention discloses a method for the production of a recombinant protein consisting of the enzyme horseradish peroxidase (HRP) fused to GBP. Many biological processes of interest generate peroxide that can provide the basis of clinical testing. Nature provides enzymes, i.e., peroxidases, to destroy cytotoxic peroxide. The electrons formed by peroxidase activity can produce an electrical current at a nearby electrode. High sensitivity can be achieved in assays of certain redox reactions using HRP fused to GBP to construct biosensing electrodes. For examples, the invention can be used to construct amperometric enzyme electrodes or other devices for the detection of hydrogen peroxide, organic hydroperoxides, phenols, aromatic amines and hazardous compounds, e.g., potassium cyanide.

Electron transfer between HRP and electrode is slow and inefficient. This is mainly attributed to the poor and random binding of native, glycosylated HRP to electrodes (Ferapontova, et. al., Biosens Bioelectron 16:147-157, 2001). In recent years, considerable effort has been made to improve the binding of HRP to gold. Genetically engineered variants of the enzyme expressed in E. coli have improved binding to gold electrodes (Ferapontova, et. al., Biosens Bioelectron 16:147-157, 2001, Ferapontova, et. al., Biosens Bioelectron 17:953-963, 2002). However, enzyme attachment occurs by random physical adsorption and, therefore, is less efficient than the controlled orientation attachment method described in the current invention. A similar recombinant HRP when adsorbed to gold electrodes was capable of direct electron transfer without the requirement of electron transfer meditors (Zeravik, et al., Biosens Bioelectron 18:1321-1327, 2003). The inventive conception, recombinant HRP-GBP fusion protein, providing controlled orientation attachment of HRP to gold electrodes can offer increased sensitivity, less electrical resistance, and greater durability to redox-based amperometric sensing and other types of electrochemical detection devices.

In another embodiment, two molecules of HRP can be produced fused to a single domain of GBP. Such a fusion protein can have greater specific activity than a recombinant molecule with only one copy of enzyme.

Those skilled in the art can produce GBP fusion proteins containing HRP by the following method: HRP can be expressed in E. coli as inclusion bodies, purified and reconstituted in vitro (Grigorenko, et. al., Biocatalysis and Biotransformation 17:359-379, 1999; Ferapontova, et. al., Biosens Bioelectron 17:953-963, 2002; and Levy, et. al., Biotechnol Bioeng 82:223-231, 2003). Fusion protein constructs can be built as instructed in Example 1 above, placing GBP either at the N-terminus or the C-terminus of HRP with a terminal His₆-tag at the same end as GBP in each case. A flexible linker sequence consisting of glycyl-seryl repeating sequences separates the HRP coding sequence from GBP. These plasmids can be expressed in the cytosol of E. coli, purified from inclusion bodies by Nickel-resin chromatography, and refolded in vitro. The coding sequence for HRP (GenBank Accession # J05552) can be obtained from researchers or synthesized from oligonucleotides.

In another embodiment, cytochrome c peroxidase that is also used for derivatizing electrodes (Ruzgas, et. al., Analytica Chimica Acta 330:123-138,1996) can be fused to GBP. The yeast enzyme has been successfully expressed in E. coli (eske, et al., Protein Expr Purif 19:139-147, 2000).

### EXAMPLE 11

### Production of Recombinant Proteins Consisting of an Enzyme, Glucose Oxidase, Fused to GBP

In one embodiment, the disclosure provides a method to produce a recombinant protein containing the enzyme glucose oxidase (GOD) fused to GBP. The invention, herein, provides benefits in the design of miniature glucose monitoring devices or other devices incorporating nanotechnology by attaching glucose oxidase to a gold surface in an efficient, low cost method providing full retention of enzyme activity and other properties.

In another embodiment, two molecules of GOD can be produced fused to a single domain of GBP. Such a fusion protein can have greater specific activity than a recombinant molecule with only one copy of enzyme.

We disclose, herein, how the invention can be applied to glucose monitoring:

The oxidation of glucose by glucose oxidase and reduction of O₂ to H₂O₂ can provide a measurable electrical current at a nearby conducting electrode, proportional to the concentration of glucose in the sample. Most commercial glucose monitoring devices operate on this principle. However, existing methods for immobilizing glucose oxidase do not allow the sensitivity envisioned for future devices employing nanotechnology designed for non-invasive testing or more accurate testing. Further, the performance of is electrical devices that measure levels of blood glucose can be diminished by irrelevant substances fouling the electrode surface. As we established in Examples 4 through 6 above, facilitated protein binding to gold via a GBP domain can significantly improve the attachment of active fusion partners and resist surface fouling compared to conventional methods employing native proteins. The disclosed invention provides similar benefits for the controlled attachment of glucose oxidase in designing improved glucose monitors and in the design of novel miniature nanodevices using gold electrodes for the purpose of detection.

Those skilled in the art can produce GBP fusion proteins as instructed in Example 1 above, containing GOD by the following method: GOD from Aspergillus niger is a dimer of molecular weight 150,000 containing two tightly bound FAD cofactors. It has been extensively used as the basis for biosensors, in glucose detection kits and as a source of hydrogen peroxide in the food industry. It has been expressed and secreted in copious amounts from yeast using either its own signal sequence or the alpha-factor leader sequence of Saccharomyces cerevisiae (Frederick, et al., J Biol Chem 265:3793-3802, 1990, Park, et al., J Biotechnol 81:35-44, 2000). It has also been secreted from S. cerevisiae with a His₆-tag at the C-terminus (Ko, et al., Protein Expr Purif 25:488-493, 2002).

The coding sequence for A. niger GOD (GenBank Accession # J05242) can be obtained from researchers or cloned by PCR from the organism. GBP can be linked to the C-terminus of GOD with a flexible spacer sequence followed by a His₆-tag. Using one of the coli-yeast shuttle vectors (Invitrogen) the fusion protein can be secreted utilizing its own signal sequence. The host strain of S. cerevisiae carries the appropriate auxotrophic markers for maintaining the plasmid and a pep4 mutation can be used to reduce protein degradation. The fusion protein can be purified from the culture medium using Nickel-resin column chromatography.

The advantage of this yeast expression strategy is that it can produce GOD-GBP in a soluble and active form in large amounts. GBP has numerous serine and threonine residues that could potentially serve as targets for O-linked glcosylation, thus masking gold binding. The electrical communication between GOD and the electrode and thereby its biosensor performance is hampered by the protein-bound carbohydrate moiety of the enzyme (Alvarez-Icaza, et al., Biosens Bioelectron 10:735-742, 1995). A pmr1 host mutation can help in this regard although with an overall inhibition of growth (Ko, et al., Protein Expr Purf 25:488-493, 2002).

In another embodiment, and to circumvent the potential glycosylation problems mentioned above for yeast-secreted GOD, glucose oxidase from Penicillium amagasakiense can be expressed without carbohydrate in the cytoplasm of E. coli. Further, GOD from P. amagasakiense has a higher turnover rate and a higher affinity for glucose than its A. niger counterpart (Kiess, et al., Eur J Biochem 252:90-99, 1998).

The coding sequence can be cloned by PCR amplification with genomic DNA from the organism as template. GBP-fusion protein constructs can be built placing GBP either at the N-terminus or the C-terminus of GOD with a terminal His6-tag at the same end as GBP in each case. A flexible linker sequence consisting of glycyl-seryl repeats can separate the GOD gene from GBP. The GOD-GBP fusion proteins can be expressed to form cytoplasmic inclusion bodies and the protein can be purified by Nickel-resin chromatography and subsequently refolded in the presence of FAD cofactor (Witt, et al., Appl Environ Microbiol 64:1405-1411, 1998).

### EXAMPLE 12

### Production of Recombinant Proteins Consisting of GBP, the Enzyme Horseradish Peroxidase, and the Enzyme Glucose Oxidase

The enzymes glucose oxidase and horseradish peroxidase can be used in combination to construct a glucose monitor that has greater sensitivity than one constructed with glucose oxidase alone. Appropriate GBP- and enzyme-containing fusion proteins can provide superior activity in enzyme-based applications compared to available enzymes currently in use.

In one embodiment, GBP-fusion partners can be horseradish peroxidase or cytochrome c peroxidase or related peroxidase and glucose oxidase or related enzyme. An advantage of such a fusion protein is to allow a significant increase in the efficiency of activity of each enzyme in enzyme electrodes, e.g. a monitor to measure blood glucose levels. Existing monitoring devices can employ both enzymes in a coupled system to provide enhanced transfer of electrons to an electrode. However, the controlled binding of enzymes provided by the current invention can result in improved efficiency compared to conventional methods to attach enzymes to electrodes.

Appropriate expression vectors can be constructed using methods described in Examples 1, 10 and 11.

### EXAMPLE 13

### Production of GBP-Containing Recombinant Proteins Consisting of Different Enzymes or Consisting of Combinations of Enzymes and Affinity Binding Polypeptides

GBP-containing fusion proteins can be produced that contain any two different enzymes, or one enzyme and a single-chain antibody, or one enzyme and any polypeptide with affinity for the substrate or product of the enzyme fusion partner.

In one embodiment, the different GBP-fusion partners can be horseradish peroxidase or related peroxidase and any oxidative enzyme. An advantage of such fusion proteins is to couple the electron-enhancing function of HRP and the like to the activity of any oxidative enzyme used to detect certain analytes.

In another embodiment, the different GBP-fusion partners can be any two enzymes or enzyme complexes with distinct activities that occur as coupled enzyme systems in nature. An advantage of such fusion proteins is to significantly enhance the overall activity of coupled enzyme systems whereby, the product of one enzyme is the substrate of the other. The close physical proximity of the two enzymes on a gold surface favors utilization of the concentrated product of the first enzyme by the second enzyme before the product can diffuse into the surrounding solution.

In another embodiment, the different GBP-fusion partners can be any two enzymes or enzyme complexes with distinct activities that do not occur as coupled enzyme systems in nature. An advantage of such fusion proteins is to provide a mechanism by which enzymes that naturally occur in uncoupled systems can be physically connected to each other and a gold surface. This allows the concentrated product of the first enzyme to be utilized as substrate of the second enzyme before the product can diffuse into the surrounding solution.

In another embodiment, the different GBP-fusion partners can be any enzyme and a scFv antibody with affinity binding activity for the substrate or product of the enzyme. An advantage of such fusion proteins is to provide a mechanism to concentrate any molecule of interest at a gold surface by binding the molecule to the fusion protein via a scFv with specificity to that molecule. A minor change in solvent conditions, e.g., increasing the salt concentration or changing the pH, can be used to release the concentrated molecule from the scFv antibody allowing the enzyme fusion partner to use the molecule as substrate. Alternatively, scFv antibodies can be selected that have relatively high dissociation constants, e.g., 104 to 10-6 M, that function to concentrate the molecule of interest from dilute solution, but with low avidity to permit relatively rapid dissociation of the molecule and allow the enzyme to utilize it as substrate.

In another embodiment, the invention discloses the production of a recombinant molecule containing GBP, a polypeptide binding a specific molecule A, and an enzyme utilizing molecule A as substrate. Such a molecule can concentrate molecule A in dilute solutions in the vicinity of the enzyme to allow a reaction not possible when all components are free in solution.

### EXAMPLE 14

### Production of Recombinant Proteins Consisting of GBP and Cell Surface Recepetors or Other Macromolecules; and Production of Recombinant Proteins Consisting of Ligands of Cell Surface Receptors or Other Macromolecules

Fusion proteins consisting of GBP and one or more copies of cell surface receptors or other surface macromolecules can have utility in constructing biodetection devices. In particular, glycosylphosphatidylinositol (GPI)-anchored cell surface proteins are widely expressed on the surface of cells, including cells of immunohematopoietic origin. The cross linking via ligand binding of GPI-anchored receptors such as Thy-1, Ly-6 A/E, CD48, CD59 and others induce a variety of T-cell activity including mitogenesis (Loertscher and Lavey, Transpl Immunol 9:93-96, 2002). Such GPI-anchored receptors are the target of intense drug discovery. GPI-anchored proteins do not contain transmembrane amino acid sequences and, therefore, ligand binding and receptor stability is not dependent on the presence of a lipid membrane. Thus, any GPI-linked protein can be a potential fusion partner with GBP for the purpose of defining ligand binding properties and screening for agonists/anatagonists of specific ligands.

In one embodiment, the invention provides for one or two copies of cell surface receptor or protein such as a GPI-anchored fusion partner for each GBP domain. The fusion protein with two copies of GPI-linked protein provides an excellent model to study the binding process of ligands that normally occurs on the surface of cells whereby ligand cross-links two GPI-linked proteins to initiate a cellular function.

In another embodiment, the invention provides a GBP-fusion protein whereby the two fusion partners are distinct GPI-linked proteins or the stable binding domain of a different type of cell surface receptor.

In another embodiment, the invention provides a GBP-fusion protein whereby the fusion partner is one or more polypeptide ligands of a cell surface receptor or other macromolecule.

### EXAMPLE 15

### Production of Recombinant Proteins Consisting of GBP and a Polypeptide Substrate(S) or a Polypeptide Inhibitor(S) of a Proteolytic Enzyme

Fusion proteins consisting of GBP and certain polypeptide substrates of proteolytic enzymes (proteases) can have utility in clinical and environmental testing. Such fusion proteins can be especially useful when utilized to support biodetection devices designed to detect protease activity in certain physiologic or environmental samples. In many instances, a determination of the presence of protease activity is a tedious process requiring the use of complex analytical equipment.

When used to support biosensors, e.g., SPR devices, fusion proteins of GBP and protease substrates can provide assays to give real-time analysis of protease activity in test samples. Further, such biosensors can function in complex solutions, e.g., crude extracts of tissues or whole blood, where the use of other types of conventional assays including coloriometric, fluorometric, or bio assays are precluded. Without limiting the scope of the invention, since there are hundreds of specific proteases that can have potential clincial, industrial and research value, a few examples are listed below.

In one embodiment, a fusion protein consisting of GBP and any of a variety of tissue collagens can be bound to a biosensing device to measure collagenase activity in tissue extracts, or cell extracts, or body fluids, or cell culture medium.

In another embodiment, a fusion protein consisting of GBP and tissue elastin can be bound to a biosensing device to measure elastase activity in tissue extracts, or cell extracts, or body fluids, or cell culture medium.

In another embodiment, a fusion protein consisting of GBP and fibrin can be bound to a biosensing device to measure fibrinolytic activity in tissue extracts, or cell extracts, or body fluids, or cell culture medium.

In another embodiment, a fusion protein consisting of GBP and any of a variety of blood coagulation factors can be bound to a biosensing device to measure the specific activity of factor activation in tissue extracts, or cell extracts, or body fluids, or cell culture medium.

In another embodiment, a fusion protein consisting of GBP and any of a variety of blood complement proteins can be bound to a biosensing device to measure the specific activity of protein activation in tissue extracts, or cell extracts, or body fluids, or cell culture medium.

In another embodiment, a fusion protein consisting of GBP and any of a variety of proteins involved in the process of apoptosis can be bound to a biosensing device to measure the specific protein activation activity in cell extracts or cell culture medium.

In another embodiment, a fusion protein consisting of GBP and a specific polypeptide substrate of a protease on or secreted from cells can be bound to a biosensing device to measure the specific protease activity on cells, or in cell extracts, or secreted by cells into culture medium or body fluids.

In another embodiment, a fusion protein consisting of GBP and a specific polypeptide substrate of a protease required for viral processing can be bound to a biosensing device to measure the specific protease activity in tissue extracts, or cell extracts, or body fluids, or in cell culture medium.

In another embodiment, a fusion protein consisting of GBP and a specific polypeptide substrate of a protease secreted from or residing on a parasite can be bound to a biosensing device to measure the specific protease activity in tissue extracts, or cell extracts or body fluids, or in cell culture medium.

In many other embodiments, a fusion protein consisting of GBP and a specific polypeptide inhibitor(s) of a protease can be bound to a biosensing device to detect the presence of a protease in test samples. The device can be used to quantify protease levels in tissue extracts, plant extracts, parasite extracts, cell extracts, body fluids, or in cell culture medium.

### EXAMPLE 16

### Binding of Recombinant GBP Fusion Proteins to Colloidal Gold and Lateral Flow Applications Thereof

Relatively few native proteins bind well to colloidal gold using standard procedures with retention of activity. The presence of salt prevents protein binding to colloidal gold and many proteins are insoluble or bind other surfaces under low salt conditions. Also, protein binding to colloidal gold is favored at a pH close to the pI of the molecule, but for many proteins of interest poor solubility occurs near the pI. Further, few small peptides of interest bind colloidal gold directly and, therefore, many potential clinical and other testing applications are currently impossible or problematic. An inventive aspect of our technology is the transfer of gold binding of any fusion polypeptide to the GBP domain regardless of the intrinsic binding affinity of its partner and under conditions, i.e., pH 7 and moderate salt concentration, that favors retention of activity and solubility of fusion proteins. An additional inventive aspect of our approach is the use of significantly less protein to saturate gold surfaces. Binding of IgG to gold requires 500 times more protein compared to that for GBP-SEAP to saturate the binding capacity.

In one embodiment, we describe the binding of the protein A- and streptavidin-GBP fusion proteins to colloidal gold under conditions instructed by Examples 4 and 5. Optimization of binding includes modification of pH, salt concentration and other variables to establish preferred GBP binding conditions. Protein A- or streptavidin-GBP binding and stability are measured using an enzymatic binding assay in which protein A and streptavidin is measured through it's ability to bind enzyme conjugated antibody.

In another embodiment, we establish a lateral flow immunodection system based on our colloidal gold binding technology (Ketema, et al., J. Acquir Immune Defic Syndr 27:63-70, 2001). One of the key market applications for colloidal gold is as a detection reagent for immunodetection in lateral flow dipstick assays. Lateral flow tests are used for the specific qualitative or semi-quantitative detection of many analytes including antigens, antibodies, and even the products of nucleic acid amplification tests. One or several analytes can be detected simultaneously on the same strip. Urine, saliva, serum, plasma, or whole blood can be used as specimens. Extracts of patient exudates or fluids have also been successfully used.

The assay uses GBP-protein A bound to colloidal gold as a detection reagent. Samples contain or lacking IgG are placed on the absorption pad, and flow with the protein A conjugate. The presence of antibody in the test solution interferes with protein A binding to the IgG test strip, but develops a band at the anti-protein A control strip. In the absence of antibody protein A binds the IgG strip, and a band is visible. Streptavidin-GBP is used in similar fashion with biotinylated targets.

1. A DNA plasmid encoding a fusion protein comprising all of, or a combination of the following components;
a gold-binding polypeptide (GBP) that can have 1 to 7 repeats of the amino acid sequence: Met-His-Gly-Lys-Thr-Gln-Ala-Thr-Ser-Gly-Thr-Ile-Gln-Ser, wherein the last repeat can have an isoleucine substituted for a threonine in the fifth position; or
a gold-binding peptide with a different amino acid sequence;
one or more polypeptide fusion partners conferring specific activities to a fusion protein;
repeating sequences of Gly-Ser of varying length to provide flexible linkers between fusion partners;
specific affinity-binding sequence such as polyhistidine, or V5 epitope, or FLAG epitope, or the like to facilitate purification of fusion proteins; and
specific peptide bonds that can be selectively hydrolyzed by enzymes or by chemical reactions.

2. The method of claim 1, wherein the DNA encodes GBP and a polypeptide fusion partner that has specific binding activity for another molecule; said fusion protein configured as polypeptide 1-GBP or GBP-polypeptide 1, and fusion partner domains separated by flexible linking sequences.

3. The method of claim 1, wherein the DNA encodes two or more copies of a distinct polypeptide fusion partner configured as polypeptide 1-GBP-polypeptide 1, and fusion partner domains separated by flexible linking sequences.

4. The method of claim 1, wherein the DNA encodes at least one copy of a distinct fusion partner and one copy of a different fusion partner configured as polypeptide 1-GBP-polypeptide 2 or polypeptide 2-GBP-polypeptide 1, and fusion partner domains separated by flexible linking sequences.

5. The method of claim 2, wherein the DNA encodes protein A, or protein G, or related molecule as a polypeptide fusion partner as in protein A-GBP or GBP-protein A.

6. The method of claim 2, wherein the DNA encodes streptavidin, or avidin, or related molecule as a polypeptide fusion partner as in streptavidin-GBP or GBP-streptavidin.

7. The method of claim 1, wherein the DNA encodes two or more copies of GBP as in GBP-GBP, or GBP-GBP-GBP etc, and the GBP domains are separated by flexible linking sequences.

8. The method of claim 3, wherein the DNA encodes at least one copy of protein A, or protein G, or related molecule fused to the amino-terminus of GBP and at least one other copy of protein A, or protein G, or related molecule fused to the carboxyl-terminus of GBP.

9. The method of claim 3, wherein the DNA encodes at least one copy of streptavidin, or avidin, or related molecule fused to the amino-terminus of GBP and at least one other copy of streptavidin, or avidin, or related molecule fused to the carboxyl-terminus of GBP.

10. The method of claim 4, wherein the DNA encodes at least one copy of protein A, or protein G, or related molecule and one copy of streptavidin, or avidin, or related molecule as polypeptide fusion partners as in protein A-GBP-streptavidin or streptavidin-GBP-protein A.

11. The method of claim 1, wherein the DNA encodes polypeptide fusion partners that are enzymes.

12. The methods of claims 1, 2, 3, and 11, wherein the DNA encodes the enzyme horseradish peroxidase (HRP) or related enzyme as polypeptide fusion partners as in HRP-GBP, or GBP-HRP, or HRP-GBP-HRP.

13. The methods of claims 1, 2, 3 and 11, wherein the DNA encodes the enzyme glucose oxidase (GOD) or related enzyme as polypeptide fusion partners as in GOD-GBP, or GBP-GOD, or GOD-GBP-GOD

14. The methods of claims 1 and 4, wherein the DNA encodes the enzyme horseradish peroxidase (HRP) or related enzyme, and the enzyme glucose oxidase (GOD) or related enzyme as polypeptide fusion partners as in HRP-GBP-GOD, or GOD-GBP-HRP.

15. The methods of claims 1 and 2, wherein the DNA encodes a polypeptide substrate or polypeptide inhibitor of a proteolytic enzyme as a fusion partner.

16. The method of claim 1, wherein the DNA encodes polypeptide fusion partners that are single-chain antibodies.

17. The method of claim 1, wherein the DNA encodes polypeptide fusion partners that are cell surface receptors, or other cell surface proteins, or ligands of cell surface receptors or proteins.

18. A method, wherein the DNA of claims 1 through 17 are expressed in bacteria, yeast, baculovirus, other microorganisms, plant cells, plants, mammalian cells or animals to produce stable and active fusion proteins containing GBP.

19. The method of claim 18, wherein the GBP-containing fusion proteins are purified by conventional means or using a polyhistidine sequence or other affinity tag sequence.

20. The method of claim 19, wherein purified GBP-containing fusion proteins are used in all fields that utilize gold.

21. The method of claim 19, wherein purified GBP-containing fusion proteins are used in biosensor or biodetection applications.

22. The method of claim 19, wherein purified GBP-containing fusion proteins are used to construct surface plasmon resonance sensors.

23. The method of claim 19, wherein purified GBP-containing fusion proteins are used to construct piezoelectric quartz crystal sensors.

24. The method of claim 19, wherein purified GBP-containing fusion proteins are used to construct amperometric electrodes.

25. The method of claim 19, wherein the produced GBP-containing fusion proteins are used in all applications utilizing colloidal gold.
